(19) United States
(12) Patent Application Publication
(10) Pub. No.: US 2026/0317369 A1
(43) Pub. Date: Oct. 15, 2026

(54) PRECISE LATE-STAGE MODIFICATION ON TRYPTOPHAN RESIDUE OF NATIVE PEPTIDES

(71) Applicant: UNIVERSITY OF HONG KONG VERSITECH LIMITED, Hong Kong (CN)

(72) Inventors: Xuechen LI, Hong Kong (CN); Yisa XIAO, Hong Kong (CN)

(21) Appl. No.: 19/482,791

(22) PCT Filed: May 7, 2024
(86) PCT No.: PCT/CN2024/091317

§ 371 Date: Nov. 10, 2025

Related U.S. Application Data
(60) Provisional application No. 63/503,068, filed on May 18, 2023.

(51) Int. Cl. C07K 1/107; C07K 7/06; C07K 7/64; C07K 14/435; C07K 14/605; C07K 14/655; C07K 14/665

(52) U.S. Cl. CPC C07K 1/1077; C07K 7/06; C07K 7/64; C07K 14/43572; C07K 14/605; C07K 14/655; C07K 14/665

(57) ABSTRACT

Methods for late-stage modification of peptides and functionalized peptides are described herein. The methods are specific and robust, allowing precise construction of C2-sulfenylated tryptophan via a sulfenyl reagent, such as a thiosulfonate (ArSO₂SR) reagent. The simple C2-sulfenylation process allows easy installation of diverse functional groups, such as SCF₃, SCF₂H, SCF₂Br, SCHFCl, SCF₂-alkyl, SCHF-alkyl, S-alkyl, S-aryl, etc., onto tryptophan residue (s) of a peptide, in particular a native peptide. Generally, the late-stage peptide modification method includes: (i) maintaining a reaction mixture at a sufficient temperature for a sufficient period of time to form a product. The reaction mixture contains the peptide being modified, which contains one or more tryptophan residues; a sulfynyl reagent; and a solvent. The product formed in step (i) contains a sulfenylated peptide, where the sulfenylation is on at least one of the one or more tryptophan residues of the peptide.

### CROSS-REFERENCE TO RELATED APPLICATION

This application claims the benefit of and priority to U.S. Provisional Application No. 63/503,068 filed May 18, 2023, the entire content of which is incorporated herein by reference for all purpose in its entirety.

### FIELD OF THE INVENTION

This invention is generally in the field of peptide modification.

### BACKGROUND OF THE INVENTION

Peptide drugs have gained significant attention in recent years due to their high specificity and efficacy in treating various diseases. However, the short half-life and low stability of these drugs in the body limit their therapeutic potential. To overcome these limitations, late-stage modifications of peptide drugs have been developed, which involve chemical or enzymatic modifications of the peptide structure to enhance their stability, bioavailability, and pharmacokinetic properties. These modifications can improve the drug's efficacy, reduce dosing frequency, and increase patient compliance.

Late-stage modification as a unique strategy to improve natural active peptides metabolic stability, membrane permeability, pharmacokinetics, and pharmacodynamics through chemical modification on specific residue which gradually becomes an effective strategy in the peptide drug discovery and development in the last two decade. However, multiple site residues of peptides make chemical selectivity an unavoidable challenge in the late-stage modification. To the date, hundreds of modification methods based on two reactive residue cysteine and lysine have been developed. Apart from this, tryptophan could be used as special site for late-stage modification due to its rare abundance (~1%) and wide distribution of peptides and proteins. Late-stage approaches for tryptophan modification have been gradually developed in the last decade years including C—H activation, radical reaction, indole dearomatization and oxidative reaction.

Albeit these developments, problems exist, for example, the metal catalyst C—H activation would generate an unavoidable drawback of trace metal residue and the radical reaction-based modification has selectivity problem between tryptophan and other side residue (like Tyrosine and Cysteine).

There is a need for methods for late-stage modification of peptides.

Therefore, it is the object of the present invention to provide a method for late-stage modification of peptides, such as late-stage modification on tryptophan residues of peptides.

It is a further object of the present invention to provide functionalized peptides, in particular peptides with sulfenyl functional groups on tryptophan residues.

### SUMMARY OF THE INVENTION

Methods for late-stage modification of peptides and functionalized peptides are described herein. The disclosed methods are specific and robust, allowing precise construction of C2-sulfenylated tryptophan via a sulfenyl reagent, such as a thiosulfonate (ArSO₂SR) reagent. The simple C2-sulfenylation process allows easy installation of diverse functional groups, such as SCF₃, SCF₂H, SCF₂Br, SCHFCl, SCF₂-alkyl, SCHF-alkyl, S-alkyl, S-aryl, etc., onto tryptophan residue(s) of a peptide, in particular a native peptide. For example, late-stage modification of peptide using the disclosed method can anchor SCF₃, SCF₂H, SCF₂Br, SCHFCl, SCF₂-alkyl, and/or SCHF-alkyl function groups onto therapeutic peptides, which is unprecedented. The disclosed methods provide an effective strategy as a toolbox for late-stage modification of peptide drugs, featuring operational simplicity and diverse product structures that were previously unobtainable.

Generally, the late-stage peptide modification method disclosed herein includes: (i) maintaining a reaction mixture at a sufficient temperature for a sufficient period of time to form a product. The reaction mixture contains the peptide being modified, which contains one or more tryptophan residues; a sulfynyl reagent; and a solvent. The product formed in step (i) contains a sulfenylated peptide, where the sulfenylation is on at least one of the one or more tryptophan residues of the peptide.

The peptide being modified using the disclosed method can be any suitable peptide, as long as it contains one or more tryptophan residue. For example, the peptide being modified can be a linear peptide or a cyclic peptide (including monocyclic, bicyclic, etc.). For example, the peptide being modified can be a random peptide or a peptide drug. In some forms, the peptide being modified using the disclosed method is only formed by natural amino acids.

In some forms, the sulfynyl reagent can be a thiosulfonate reagent having the structure of:

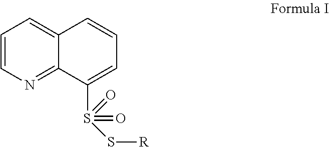

wherein R can be a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, can be a halogen, a hydroxyl, an azido, an alkyl, an alkynyl, an alkoxy, a carbonyl (e.g., a carboxyl), a nitro, or a cyano.

The solvent can be any suitable solvent that dissolves the peptide and the sulfynyl reagent. For example, the solvent used to form the reaction mixture is trifluoroacetic acid. In some forms, when trifluoroacetic acid is used, it can also act as a catalyst for the reaction in step (i) and/or be the only solvent in the reaction mixture.

The reaction between the peptide and the sulfynyl reagent in step (i) can be performed at room temperature (i.e., 20° C. to 25° C., at 1 atm) or a temperature that is close to room temperature (e.g., up to 35° C., at 1 atm), for a time period sufficient to form the product that contains the sulfenylated peptide. For example, the reaction in step (i) is performed at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

In some forms, the sulfenylated peptide formed from the reaction between the peptide and the sulfynyl reagent in step (i) contains one or more sulfenylated tryptophan residues represented by

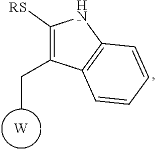

wherein R can be a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, can be a halogen, a hydroxyl, an azido, an alkyl, an alkynyl, an alkoxy, a carbonyl (e.g., carboxyl), a nitro, or a cyano.

### BRIEF DESCRIPTION OF THE DRAWINGS

FIG. 1 is a graph showing the UV (190-400 nm) and MS (250-3000 m/z) traces from UPLC-MS analysis of 2a (gradient 5-95% CH₃CN/H₂O containing 0.1% TFA over 5 min at a flow rate of 0.4 mL/min).

FIG. 2 is a graph showing the UV (190-400 nm) and MS (250-3000 m/z) traces from UPLC-MS analysis of 2j (gradient 5-95% CH₃CN/H₂O containing 0.1% TFA over 5 min at a flow rate of 0.4 mL/min).

FIG. 3 is a graph showing the UV (190-400 nm) and MS (250-3000 m/z) traces from UPLC-MS analysis of 21 (gradient 5-95% CH₃CN/H₂O containing 0.1% TFA over 5 min at a flow rate of 0.4 mL/min).

FIG. 4 is a graph showing the UV (190-400 nm) and MS (250-3000 m/z) traces from UPLC-MS analysis of 2q (gradient 5-95% CH₃CN/H₂O containing 0.1% TFA over 5 min at a flow rate of 0.4 mL/min).

FIG. 5 is a graph showing the UV (190-400 nm) and MS (250-3000 m/z) traces from UPLC-MS analysis of 21 (gradient 5-95% CH₃CN/H₂O containing 0.1% TFA over 5 min at a flow rate of 0.4 mL/min).

FIG. 6 is a graph showing the UV (190-400 nm) and MS (250-3000 m/z) traces from UPLC-MS analysis of 3a (gradient 5-95% CH₃CN/H₂O containing 0.1% TFA over 5 min at a flow rate of 0.4 mL/min).

FIG. 7 is a graph showing the UV (190-400 nm) and MS (250-3000 m/z) traces from UPLC-MS analysis of 3d (gradient 5-95% CH₃CN/H₂O containing 0.1% TFA over 5 min at a flow rate of 0.4 mL/min).

FIG. 8 is a graph showing the UV (190-400 nm) and MS (300-2000 m/z) traces from UPLC-MS analysis of 3e (gradient 5-95% CH₃CN/H₂O containing 0.1% TFA over 5 min at a flow rate of 0.4 mL/min).

FIG. 9 is a graph showing the UV (190-400 nm) and MS (250-3000 m/z) traces from UPLC-MS analysis of 3f (gradient 5-95% CH₃CN/H₂O containing 0.1% TFA over 5 min at a flow rate of 0.4 mL/min).

FIG. 10 is a graph showing the UV (190-400 nm) and MS (250-3000 m/z) traces from UPLC-MS analysis of 3g (gradient 5-95% CH₃CN/H₂O containing 0.1% TFA over 5 min at a flow rate of 0.4 mL/min).

FIG. 11 is a graph showing the UV (190-400 nm) and MS (250-3000 m/z) traces from UPLC-MS analysis of 3k (gradient 5-95% CH₃CN/H₂O containing 0.1% TFA over 5 min at a flow rate of 0.4 mL/min).

FIG. 12 is a graph showing the UV (190-400 nm) and MS (250-3000 m/z) traces from UPLC-MS analysis of 31 (gradient 5-95% CH₃CN/H₂O containing 0.1% TFA over 5 min at a flow rate of 0.4 mL/min).

FIG. 13 is a graph showing the UV (190-400 nm) and MS (250-3000 m/z) traces from UPLC-MS analysis of 3m (gradient 5-95% CH₃CN/H₂O containing 0.1% TFA over 5 min at a flow rate of 0.4 mL/min).

FIG. 14 is a graph showing the UV (190-400 nm) and MS (250-3000 m/z) traces from UPLC-MS analysis of 3t (gradient 5-95% CH₃CN/H₂O containing 0.1% TFA over 5 min at a flow rate of 0.4 mL/min).

FIG. 15 is a graph showing the UV (190-400 nm) and MS (250-3000 m/z) traces from UPLC-MS analysis of 3u (gradient 5-95% CH₃CN/H₂O containing 0.1% TFA over 5 min at a flow rate of 0.4 mL/min).

### DETAILED DESCRIPTION OF THE INVENTION

Late-stage modification of tryptophan residues of peptides using previous approaches suffers from contaminant by trace metal residue used in the modification reactions, long reaction time and/or elevated temperature for reactions, and selectivity problem between tryptophan residue and other side residues (like Tyrosine and Cysteine).

The disclosed method uses a sulfenyl reagent to achieve precise construction of C2-sulfenylated tryptophan of peptides. As such, the disclosed method allows for full conversion of the tryptophan residue within 2 hours or within 1 hour at room temperature (i.e., 20° C.-25° C. at 1 atm) or a temperature (e.g., up to about 35° C. at 1 atm, such as about 30° C.) close to room temperature without racemic problem, is tolerant to 19 amino acid residues (for example, the disclosed method uses non-oxidative/reductive condition and thus is friendly to oxidation-sensitive side residue such as lysine), avoids generation of trace metal due to the metal-free reaction condition, and/or provides chemical selectivity of modification on tryptophan residue as low equivalents of reagent as possible. For example, the disclosed method only needs 1.2-5.0 equivalents reagent to drive the full conversion of tryptophan residues with great tolerance to all native amino acids.

### I. Definitions

The term “alkyl” refers to a branched or straight-chain monovalent saturated aliphatic hydrocarbon radical of one to eight carbon atoms. This term is further exemplified by radicals such as methyl, ethyl, n-propyl, isopropyl, n-butyl, s-butyl, t-butyl, n-pentyl, isopentyl, t-pentyl, n-hexyl, etc.

The term “alkenyl” refers to a branched or straight-chain hydrocarbon group of from two to eight carbon atoms and structural formula containing at least one carbon-carbon double bond.

The term “alkynyl” refers to a branched or straight-chain hydrocarbon group of from two to eight carbon atoms and structural formula containing at least one carbon-carbon triple bond.

The term “aryl” refers to C₅-C₂₀-membered aromatic or fused aromatic ring systems. Examples of aromatic groups are benzene, naphthalene, anthracene, phenanthrene, chrysene, pyrene, corannulene, coronene, etc.

The term “cyclic group” refers to a saturated or unsaturated monocyclic ring or a polycyclic ring (such as those formed from single or fused ring systems), such as a cycloalkyl, a cycloalkenyl, or a cycloalkynyl, which have from three to ten carbon atoms, as geometric constraints permit.

The term “heteroalkyl” refers to straight or branched chain carbon-containing alkyl radicals containing at least one heteroatom on the carbon backbone. Suitable heteroatoms include, but are not limited to, O, N, Si, P and S, wherein the nitrogen, phosphorous and sulfur atoms are optionally oxidized, and the nitrogen heteroatom is optionally quaternized.

The term “heteroaryl” refers to C₅-C₂₀-membered aromatic or fused aromatic ring systems, in which one or more carbon atoms on one or more aromatic ring structures have been substituted with a heteroatom. Suitable heteroatoms include, but are not limited to, oxygen, sulfur, and nitrogen. Examples of heteroaryl groups pyrrole, furan, thiophene, imidazole, oxazole, thiazole, triazole, tetrazole, pyrazole, pyridine, pyrazine, pyridazine and pyrimidine, and the like. Examples of heteroaryl rings include, but are not limited to, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, furanyl, furazanyl, imidazolidinyl, imidazolinyl, imidazolyl, 1H-indazolyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isoxazolyl, methylenedioxyphenyl, naphthyridinyl, octahydroisoquinolinyl, 1,2,3-oxadiazolyl, 1,2,4-oxadiazolyl, 1,2,5-oxadiazolyl, 1,3,4-oxadiazolyl, oxazolidinyl, oxazolyl, oxindolyl, pyrimidinyl, phenanthridinyl, phenanthrolinyl, phenazinyl, phenothiazinyl, phenoxathinyl, phenoxazinyl, phthalazinyl, pteridinyl, purinyl, pyrazinyl, pyrazolidinyl, pyrazolinyl, pyrazolyl, pyridazinyl, pyridooxazole, pyridoimidazole, pyridothiazole, pyridinyl, pyridyl, pyrimidinyl, pyrrolidinyl, pyrrolinyl, pyrrolyl, quinazolinyl, quinolinyl, quinoxalinyl, tetrahydroisoquinolinyl, tetrahydroquinolinyl, tetrazolyl, 1,2,3-thiadiazolyl, 1,2,4-thiadiazolyl, 1,2,5-thiadiazolyl, 1,3,4-thiadiazolyl, thianthrenyl, thiazolyl, thienyl, thienothiazolyl, thienooxazolyl, thienoimidazolyl, thiophenyl and xanthenyl. One or more of the rings can be substituted as defined below for “substituted heteroaryl.”

The term “heterocyclic group” refers to a cyclic group as defined above where at least one of the carbon atoms of the ring is substituted with a heteroatom, such as, nitrogen, oxygen, sulfur, or phosphorus.

The term “aralkyl” refers to an aryl group or a heteroaryl group having an alkyl, alkynyl, or alkenyl group as defined above attached to the aromatic group, such as an aryl, a heteroaryl, a polyaryl, or a polyheteroaryl. An example of an aralkyl group is a benzyl group.

The terms “alkoxyl” refers to compounds represented by the formula —OR^{v}, wherein R^{v} includes, but is not limited to, an alkyl, an alkenyl, an alkynyl, a cycloalkyl, a heterocyclic, a cycloalkenyl, an aryl, a heteroaryl, an aralkyl, a heteroalkyl, etc.

Exemplary alkoxyl groups include methoxy, ethoxy, propyloxy, tert-butoxy and the like.

A “lower alkoxyl” group is an alkoxyl group containing from one to six carbon atoms.

An “ether” is two functional groups covalently linked by an oxygen as defined below.

Accordingly, the substituent of an alkyl that renders that alkyl an ether is or resembles an alkoxyl, such as can be represented by one of —O-alkyl, —O-alkenyl, —O-alkynyl, —CO— aralkyl, —O-aryl, —O-heteroaryl, —O-cyclic, —O-heterocyclic, etc.

The term “amino” as used herein includes the group

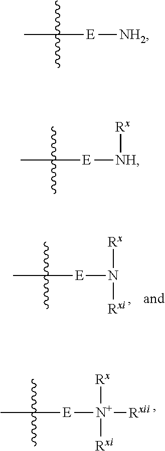

wherein, E is absent, or E is substituted or unsubstituted alkyl, substituted or unsubstituted alkenyl, substituted or unsubstituted alkynyl, substituted or unsubstituted aralkyl, substituted or unsubstituted aryl, substituted or unsubstituted heteroaryl, a substituted or unsubstituted polyaryl, a substituted or unsubstituted polyheteroaryl, substituted or unsubstituted heterocyclyl, wherein independently of E, R^{x}, R^{xi}, and R^{xii} each independently represent a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. The term “quaternary amino” also includes the groups where the nitrogen, R^{x}, R^{xi}, and R^{xii} with the N⁺ to which they are attached complete a heterocyclyl or heteroaryl having from 3 to 14 atoms in the ring structure. It is understood by those of ordinary skill in the art, that the E groups listed above are divalent (e.g., methylene, ethane-1,2-diyl, ethene-1,2-diyl, 1,4-phenylene, cyclohexane-1,2-diyl).

The terms “amide” or “amido” are used interchangeably, refer to both “unsubstituted amido” and “substituted amido” and are represented by the general formula:

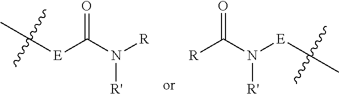

wherein, E is absent, or E is a substituted or unsubstituted alkyl, a substituted or unsubstituted alkenyl, a substituted or unsubstituted alkynyl, a substituted or unsubstituted aralkyl, a substituted or unsubstituted aryl, substituted or unsubstituted heteroaryl, a substituted or unsubstituted polyaryl, a substituted or unsubstituted polyheteroaryl, or a substituted or unsubstituted heterocyclyl, wherein independently of E, R and R′ each independently represent a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, a phosphonyl, a sulfinyl, a silyl, a thiol, an amido, an amino, or —(CH₂)ₘ—R′″, or R and R′ taken together with the N atom to which they are attached complete a heterocycle having from 3 to 14 atoms in the ring structure; R′″ represents a hydroxyl group, a substituted or unsubstituted carbonyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. In some forms, when E is oxygen, a carbamate is formed. It is understood by those of ordinary skill in the art, that the E groups listed above are divalent (e.g., methylene, ethane-1,2-diyl, ethene-1,2-diyl, 1,4-phenylene, cyclohexane-1,2-diyl).

“Carbonyl,” as used herein, is art-recognized and includes such moieties as can be represented by the general formula:

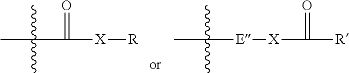

wherein X is a bond, or represents an oxygen or a sulfur, and R represents a hydrogen, a substituted or unsubstituted alkyl, a substituted or unsubstituted alkenyl, a substituted or unsubstituted alkynyl, a substituted or unsubstituted carbonyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″, or a pharmaceutical acceptable salt; E″ is absent, or E″ is a substituted or unsubstituted alkyl, a substituted or unsubstituted alkenyl, a substituted or unsubstituted alkynyl, a substituted or unsubstituted aralkyl, a substituted or unsubstituted aryl, a substituted or unsubstituted heteroaryl, a substituted or unsubstituted polyaryl, a substituted or unsubstituted polyheteroaryl, a substituted or unsubstituted heterocyclyl; R′ represents a hydrogen, a substituted or unsubstituted alkyl, a substituted or unsubstituted alkenyl, a substituted or unsubstituted alkynyl, a substituted or unsubstituted heterocyclyl, a substituted or unsubstituted aralkyl (e.g. a substituted or unsubstituted alkylaryl, a substituted or unsubstituted arylalkyl), a substituted or unsubstituted aryl, a substituted or unsubstituted heteroaryl, a substituted or unsubstituted polyaryl, a substituted or unsubstituted polyheteroaryl, a substituted or unsubstituted heterocyclyl, a hydroxyl, an alkoxy, a phosphonium, a phosphanyl, an amido, an amino, or —(CH₂)ₘ—R″; R″ represents a hydroxyl group, a substituted or unsubstituted aryl, a substituted or unsubstituted cycloalkyl, a substituted or unsubstituted cycloalkenyl, a substituted or unsubstituted heterocyclyl, a substituted or unsubstituted aryl, a substituted or unsubstituted heteroaryl, a substituted or unsubstituted polyaryl, a substituted or unsubstituted polyheteroaryl, an alkoxy, a phosphonium, a phosphanyl, an amido, or an amino; and m is zero or an integer ranging from 1 to 8. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphonium, phosphanyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, —CN, aryl, heteroaryl, and combinations thereof. It is understood by those of ordinary skill in the art, that the E″ groups listed above are divalent (e.g., methylene, ethane-1,2-diyl, ethene-1,2-diyl, 1,4-phenylene, cyclohexane-1,2-diyl). Where X is oxygen and R is defined as above, the moiety is also referred to as a carboxyl group. When X is oxygen and R is hydrogen, the formula represents a “carboxylic acid.” Where X is oxygen and R′ is hydrogen, the formula represents a “formate.” Where X is oxygen and R or R′ is not hydrogen, the formula represents an “ester.” In general, where the oxygen atom of the above formula is replaced by a sulfur atom, the formula represents a “thiocarbonyl” group. Where X is sulfur and R or R′ is not hydrogen, the formula represents a “thioester.” Where X is sulfur and R is hydrogen, the formula represents a “thiocarboxylic acid.” Where X is sulfur and R′ is hydrogen, the formula represents a “thioformate.” Where X is a bond and R is not hydrogen, the above formula represents a “ketone.” Where X is a bond and R is hydrogen, the above formula represents an “aldehyde.”

The terms “thiol” are used interchangeably and are represented by —SR, where R can be a hydrogen, a substituted or unsubstituted alkyl, a substituted or unsubstituted alkenyl, a substituted or unsubstituted alkynyl, a substituted or unsubstituted heterocyclyl, a substituted or unsubstituted aryl, a substituted or unsubstituted heteroaryl, a substituted or unsubstituted aralkyl (e.g. a substituted or unsubstituted alkylaryl, a substituted or unsubstituted arylalkyl, etc.), a substituted or unsubstituted polyaryl, a substituted or unsubstituted polyheteroaryl, a substituted or unsubstituted carbonyl, a phosphonium, a phosphanyl, an amido, an amino, an alkoxy, an oxo, a phosphonyl, a sulfinyl, or a silyl, described above. Such substituents can be any substituents described above, e.g., halogen, azide, alkyl, aralkyl, alkenyl, alkynyl, cycloalkyl, hydroxyl, carbonyl (such as a carboxyl, alkoxycarbonyl, formyl, or an acyl), silyl, ether, ester, thiocarbonyl (such as a thioester, a thioacetate, or a thioformate), alkoxyl, phosphoryl, phosphate, phosphonate, phosphinate, amino (e.g. quarternized amino), amido, amidine, imine, cyano, nitro, azido, sulfhydryl, alkylthio, sulfate, sulfonate, sulfamoyl, sulfonamido, sulfonyl, heterocyclyl, alkylaryl, haloalkyl, CN, aryl, heteroaryl, polyaryl, polyheteroaryl, and combinations thereof.

Use of the term “about” is intended to describe values either above or below the stated value, which the term “about” modifies, to be within a range of approximately +/−10%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

“Substituted,” as used herein, refers to all permissible substituents of the compounds or functional groups described herein. In the broadest sense, the permissible substituents include acyclic and cyclic, branched and unbranched, carbocyclic and heterocyclic, aromatic and nonaromatic substituents of organic compounds. Illustrative substituents include, but are not limited to, halogens, hydroxyl groups, or any other organic groupings containing any number of carbon atoms, preferably 1-14 carbon atoms, and optionally include one or more heteroatoms such as oxygen, sulfur, or nitrogen grouping in linear, branched, or cyclic structural formats. Representative substituents include a substituted or unsubstituted alkyl, a substituted or unsubstituted alkenyl, a substituted or unsubstituted alkynyl, a substituted or unsubstituted heterocyclic, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, an amino acid. Such a substituted or unsubstituted alkyl, a substituted or unsubstituted alkenyl, a substituted or unsubstituted alkynyl, a substituted or unsubstituted heterocyclyl, a substituted or unsubstituted phenyl, a substituted or unsubstituted aryl, a substituted or unsubstituted heteroaryl, a substituted or unsubstituted polyaryl, a substituted or unsubstituted polyheteroaryl, a substituted or unsubstituted aralkyl, a halogen, a hydroxyl, an alkoxy, a phenoxy, an aroxy, a silyl, a thiol, an alkylthio, a substituted alkylthio, a phenylthio, an arylthio, a cyano, an isocyano, a nitro, a substituted or unsubstituted carbonyl, a carboxyl, an amino, an amido, an oxo, a sulfinyl, a sulfonyl, a sulfonic acid, a phosphonium, a phosphanyl, a phosphoryl, a phosphonyl, and an amino acid can be further substituted.

Heteroatoms such as nitrogen may have hydrogen substituents and/or any permissible substituents of organic compounds described herein which satisfy the valences of the heteroatoms. It is understood that “substitution” or “substituted” includes the implicit proviso that such substitution is in accordance with permitted valence of the substituted atom and the substituent, and that the substitution results in a stable compound, i.e., a compound that does not spontaneously undergo transformation such as by rearrangement, cyclization, elimination, etc.

### II. Methods

Methods for late-stage modification of peptides, such as modification on the tryptophan residues of peptides, in particular native peptides, are described herein. The disclosed methods allow for precise construction of C2-sulfenylated tryptophan via a sulfenyl reagent, such as a thiosulfonate (ArSO₂SR) reagent. Thus, the methods disclosed herein provide a simple C2-sulfenylation process for easy installation of diverse functional groups, such as SCF₃, SCF₂H, SCF₂Br, SCHFCl, SCF₂-alkyl, SCHF-alkyl, S-alkyl, S-aryl, etc., onto tryptophan residue(s) of a peptide, in particular a native peptide. For example, late-stage modification of peptide using the disclosed method can install diverse sulfenyl functional groups, including SCF₃, SCF₂H, SCF₂Br, SCHFCl, SCF₂-alkyl and/or SCHF-alkyl groups, onto therapeutic peptides, which is unprecedented compared to existing methods for late-stage modification of tryptophan.

The late-stage peptide modification method disclosed herein is the first instance of using a sulfynyl reagent, such as a thiosulfonate (ArSO2SR) reagent, to selectively introduce sulfenyl functional group on tryptophan of peptides (e.g., native peptides), and thus expand the functions of peptides (e.g., native peptides). The specific and robust method disclosed herein is tolerant of all functional amino acid side chains (expect tryptophan residue, which is functionalized via the sulfynyl reagent using the method). Further, in some forms, the specific and robust late-stage modification method uses a reaction conducted under pure trifluoroacetic acid (TFA) conditions, where the TFA can act as the catalyst and sole solvent in this reaction. Compared to previous late-stage modification approaches under aqueous conditions, the disclosed method solves the problem of solubility of peptides in aqueous conditions since almost all peptide sequences can dissolve in TFA. The disclosed method also features great residue group selectivity towards tryptophan, tolerating all other functional amino acid side chains.

The sulfenylated peptides produced using the disclosed methods can have a variety of applications. For example, the disclosed method can install SCF₃, SCF₂H, SCF₂Br, SCHFCl, SCF₂-alkyl and/or SCHF-alkyl groups, onto therapeutic peptides. Fluorine atom is widely applied in drugs and agrochemicals greatly results of its unique properties of strong electronegativity (X=3.98) and small atomic radius (van der Waals radius, F: 1.47 Å) to adjust target the molecule's physicochemical properties and pharmacokinetics. Further, incorporation of fluorine into therapeutic peptides makes it also a potential diagnostic agent as sensor of “zero-background” ¹⁹FNMR. Additionally, SCF₂Br and SCHFCl-substituted reagents can introduce precursors of SCF₂¹⁸F and SHF¹⁸F for developing ¹⁸F labeling peptide therapeutic agent.

Generally, the late-stage peptide modification method disclosed herein includes: (i) maintaining a reaction mixture at a sufficient temperature for a sufficient period of time to form a product. The reaction mixture contains the peptide being modified, which contains one or more tryptophan residues; a sulfynyl reagent; and a solvent. The product formed in step (i) contains a sulfenylated peptide, where the sulfenylation is on at least one of the one or more tryptophan residues of the peptide.

The peptide and the sulfynyl reagent in the reaction mixture can have any suitable molar ratio. In some forms, the moles of sulfynyl reagent are in excess compared to the moles of the peptide. For example, the peptide and the sulfynyl reagent in the reaction mixture has a molar ratio (peptide:sulfynyl reagent) ranging from 0.1 to 1, such as about 0.2 (i.e., peptide:sulfynyl reagent=1:5).

The peptide being modified using the disclosed method can be any suitable peptide, as long as it contains one or more tryptophan residue. For example, the peptide being modified can be a linear peptide or a cyclic peptide (including monocyclic, bicyclic, etc.). For example, the peptide being modified can be a random peptide or a peptide drug. In some forms, the peptide being modified using the disclosed method is only formed by natural amino acids.

In some forms, the sulfynyl reagent can be a thiosulfonate reagent having the structure of:

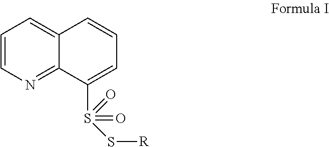

wherein R can be a haloalkyl, a substituted or unsubstituted alkyl (e.g., a substituted or unsubstituted C1-C6 alkyl), a substituted or unsubstituted alkenyl, a substituted or unsubstituted alkynyl (e.g., a substituted or unsubstituted C1-C6 alkynyl, for example, —CH₂CCH), a substituted or unsubstituted aryl (e.g., a substituted or unsubstituted phenyl), a substituted or unsubstituted polyaryl, a substituted or unsubstituted heteroaryl, a substituted or unsubstituted heteropolyaryl, a substituted or unsubstituted heterocyclic, and wherein the substituents, when present, can be a halogen (e.g., —F, —Br, —Cl, etc.), a hydroxyl, an azido, an alkyl (e.g., an unsubstituted C1-C6 alkyl, C1-C5 alkyl, C1-C4 alkyl, or C1-C3 alkyl, for example, methyl, ethyl, n-propyl, isopropyl, n-butyl, isobutyl, tert-butyl, etc.), an alkynyl (e.g., an unsubstituted C1-C6 alkynyl, C1-C5 alkynyl, C1-C4 alkynyl, or C1-C3 alkynyl, for example, a C2 alkynyl), an alkoxy, a carbonyl (e.g., carboxyl), a nitro, or a cyano.

The solvent can be any suitable solvent that dissolves the peptide and the sulfynyl reagent. For example, the solvent used to form the reaction mixture is trifluoroacetic acid. In some forms, when trifluoroacetic acid is used, it can also act as catalyst for the reaction in step (i) and/or be the only solvent in the reaction mixture.

The reaction between the peptide and the sulfynyl reagent in step (i) can be performed at room temperature (i.e., 20° C. to 25° C., at 1 atm) or a temperature that is close to room temperature (e.g., up to 35° C., at 1 atm), for a time period sufficient to form the product that contains the sulfenylated peptide. For example, the reaction in step (i) is performed at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

In some forms, the sulfenylated peptide formed from the reaction between the peptide and the sulfynyl reagent in step (i) contains one or more sulfenylated tryptophan residues represented by

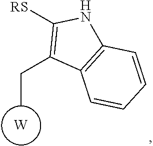

wherein R can be as defined above for Formula I.

In some forms, the peptide being modified using the disclosed method can have the structure of:

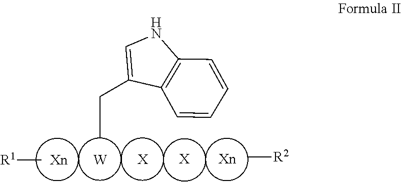

and the sulfenylated peptide formed therefrom can have the structure of:

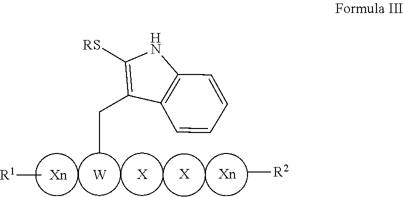

wherein each n can be independently 0 or a positive integer (such as an integer from 1 to 50, from 1 to 40, from 1 to 30, from 1 to 20, from 1 to 10, from 1 to 6, from 1 to 5, from 1 to 4, from 1 to 3, from 1 to 2, or 1); R₁ (R¹ and R₁ are used interchangeably herein) can be an amino group (such as —NH₂ or NHAc); R₂ (R² and R₂ are used interchangeably herein) can be a carbonyl group (such as —COOH) or an amido group (such as —CONH₂); each X can be independently a natural amino acid residue (including L- and D-configurations); R can be as defined above for Formula I; and

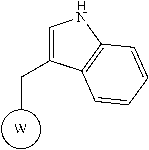

represents a L- or D-tryptophan residue. In some forms, X is not a tryptophan residue.

In some forms, the peptide being modified using the disclosed method can be a peptide drug, and the sulfenylated peptide formed therefrom can be the peptide drug with one or more sulfenylated tryptophan residues represented by

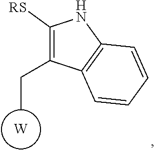

wherein R can be as defined above for Formula I.

The peptide drug being modified using the disclosed method can be any suitable peptide, linear or cyclic, as long as it contains one or more tryptophan residues. For example, the peptide drug is octreotide, lanreotide, setmelanotide, somatostain, semaglutide, leuprolide, histrelin, triptorelin, goserelin, melltin, or daptomycin.

In some forms, for any of the structures described above, the R group can be —CF₃, —CF₂H, —CF₂Br, —CHFCl, —CF₂-alkyl, —CHF-alkyl, a substituted or unsubstituted alkyl (e.g., a substituted or unsubstituted C1-C6 alkyl, a substituted or unsubstituted C1-C5 alkyl, a substituted or unsubstituted C1-C4 alkyl, or a substituted or unsubstituted C1-C3 alkyl), a substituted or unsubstituted alkynyl (e.g., —CH₂CCH), or a substituted or unsubstituted aryl (e.g., a substituted or unsubstituted phenyl), and wherein the substituent(s), when present, is a halogen, a hydroxyl, a carboxyl, an azido, an alkyl (e.g., an unsubstituted C1-C6 alkyl, for example, methyl, ethyl, propyl, or butyl), an alkynyl, or an alkoxy.

In some forms, for any of the structures described above, the R group can be —CF₃, —CF₂H, —CF₂Br, —CHFCl, —CF₂-alkyl, —CHF-alkyl, a substituted or unsubstituted C1-C6 or C1-C5 alkyl, a substituted or unsubstituted C1-C6 or C1-C5 alkynyl (e.g., —CH₂CCH), or a substituted or unsubstituted phenyl, wherein the substituent(s), when present, is a halogen, a hydroxyl, a carboxyl, an azido, an alkyl (e.g., an unsubstituted C1-C6 alkyl, for example, methyl, ethyl, propyl, or butyl), or an alkynyl (e.g., an unsubstituted C2-C6 alkynyl, for example, an unsubstituted C2 alkynyl).

When halogen(s) is/are contained in any of the structures described above, it can be an isotope, such as ¹⁸F. Alternatively or additionally, other atoms in any of the structures described herein may be isotopes as well, such as deuterium.

When alkyl is present in any of the structures described above, the alkyl can be independently a linear alkyl, a branched alkyl, or a cyclic alkyl (either monocyclic or polycyclic), substituted or unsubstituted. Exemplary alkyl include a linear C₁-C₃₀ alkyl, a branched C₄-C₃₀ alkyl, a cyclic C₃-C₃₀ alkyl, a linear C₁-C₂₀ alkyl, a branched C₄-C₂₀ alkyl, a cyclic C₃-C₂₀ alkyl, a linear C₁-C₁₀ alkyl, a branched C₄-C₁₀ alkyl, a cyclic C₃-C₁₀ alkyl, a linear C₁-C₆ alkyl, a branched C₄-C₆ alkyl, a cyclic C₃-C₆ alkyl, a linear C₁-C₄ alkyl, cyclic C₃-C₄ alkyl, such as a linear C₁-C₁₀, C₁-C₉, C₁-C₈, C₁-C₇, C₁-C₆, C₁-C₅, C₁-C₄, C₁-C₃, or C₁-C₂ alkyl group, a branched C₃-C₉, C₃-C₉, C₃-C₈, C₃-C₇, C₃-C₆, C₃-C₅, or C₃-C₄ alkyl group, or a cyclic C₃-C₉, C₃-C₉, C₃-C₈, C₃-C₇, C₃-C₆, C₃-C₅, or C₃-C₄ alkyl group. The cyclic alkyl can be a monocyclic or polycyclic alkyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkyl group.

When alkenyl is present in any of the structures described above, the alkenyl can be independently a linear alkenyl, a branched alkenyl, or a cyclic alkenyl (either monocyclic or polycyclic), substituted or unsubstituted. Exemplary alkenyl include a linear C₂-C₃₀ alkenyl, a branched C₄-C₃₀ alkenyl, a cyclic C₃-C₃₀ alkenyl, a linear C₂-C₂₀ alkenyl, a branched C₄-C₂₀ alkenyl, a cyclic C₃-C₂₀ alkenyl, a linear C₂-C₁₀ alkenyl, a branched C₄-C₁₀ alkenyl, a cyclic C₃-C₁₀ alkenyl, a linear C₂-C₆ alkenyl, a branched C₄-C₆ alkenyl, a cyclic C₃-C₆ alkenyl, a linear C₂-C₄ alkenyl, cyclic C₃-C₄ alkenyl, such as a linear C₂-C₁₀, C₂-C₉, C₂-C₈, C₂-C₇, C₂-C₆, C₂-C₅, C₂-C₄, C₂-C₃, or C₂ alkenyl group, a branched C₃-C₉, C₃-C₉, C₃-C₅, C₃-C₇, C₃-C₆, C₃-C₅, or C₃-C₄ alkenyl group, or a cyclic C₃-C₉, C₃-C₉, C₃-C₈, C₃-C₇, C₃-C₆, C₃-C₅, or C₃-C₄ alkenyl group. The cyclic alkenyl can be a monocyclic or polycyclic alkenyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkenyl group.

When alkynyl is present in any of the structures described above, the alkynyl can be independently a linear alkynyl, a branched alkynyl, or a cyclic alkynyl (either monocyclic or polycyclic), substituted or unsubstituted. Exemplary alkynyl include a linear C₂-C₃₀ alkynyl, a branched C₄-C₃₀ alkynyl, a cyclic C₃-C₃₀ alkynyl, a linear C₂-C₂₀ alkynyl, a branched C₄-C₂₀ alkynyl, a cyclic C₃-C₂₀ alkynyl, a linear C₂-C₁₀ alkynyl, a branched C₄-C₁₀ alkynyl, a cyclic C₃-C₁₀ alkynyl, a linear C₂-C₆ alkynyl, a branched C₄-C₆ alkynyl, a cyclic C₃-C₆ alkynyl, a linear C₂-C₄ alkynyl, cyclic C₃-C₄ alkynyl, such as a linear C₂-C₁₀, C₂-C₉, C₂-C₈, C₂-C₇, C₂-C₆, C₂-C₅, C₂-C₄, C₂-C₃, or C₂ alkynyl group, a branched C₃-C₉, C₃-C₉, C₃-C₅, C₃-C₇, C₃-C₆, C₃-C₅, or C₃-C₄ alkynyl group, or a cyclic C₃-C₉, C₃-C₉, C₃-C₈, C₃-C₇, C₃-C₆, C₃-C₅, or C₃-C₄ alkynyl group. The cyclic alkynyl can be a monocyclic or polycyclic alkynyl, such as a C₄-C₃₀, C₄-C₂₅, C₄-C₂₀, C₄-C₁₈, C₄-C₁₆, C₄-C₁₅, C₄-C₁₄, C₄-C₁₃, C₄-C₁₂, C₄-C₁₀, C₄-C₉, C₄-C₈, C₄-C₇, C₄-C₆, or C₄-C₅ monocyclic or polycyclic alkynyl group.

When aryl is present in any of the structures described above, the aryl group can be independently a C₅-C₃₀ aryl, a C₅-C₂₀ aryl, a C₅-C₁₂ aryl, a C₅-C₁₁ aryl, a C₅-C₉ aryl, a C₆-C₂₀ aryl, a C₆-C₁₂ aryl, a C₆-C₁₁ aryl, or a C₆-C₉ aryl. It is understood that the aryl can be a heteroaryl, such as a C₅-C₃₀ heteroaryl, a C₅-C₂₀ heteroaryl, a C₅-C₁₂ heteroaryl, a C₅-C₁₁ heteroaryl, a C₅-C₉ heteroaryl, a C₆-C₃₀ heteroaryl, a C₆-C₂₀ heteroaryl, a C₆-C₁₂ heteroaryl, a C₆-C₁₁ heteroaryl, or a C₆-C₉ heteroaryl.

When polyaryl is present in any of the structures described above, the polyaryl group can be independently a C₅-C₃₀ polyaryl, a C₅-C₂₀ polyaryl, a C₅-C₁₂ polyaryl, a C₅-C₁₁ polyaryl, a C₅-C₉ polyaryl, a C₆-C₂₀ polyaryl, a C₆-C₁₂ polyaryl, a C₆-C₁₁ polyaryl, or a C₆-C₉ polyaryl. It is understood that the aryl can be a heteroaryl, such as a C₅-C₃₀ heteropolyaryl, a C₅-C₂₀ heteropolyaryl, a C₅-C₁₂ heteropolyaryl, a C₅-C₁₁ heteropolyaryl, a C₅-C₉ heteropolyaryl, a C₆-C₃₀ heteropolyaryl, a C₆-C₂₀ heteropolyaryl, a C₆-C₁₂ heteropolyaryl, a C₆-C₁₁ heteropolyaryl, or a C₆-C₉ heteropolyaryl.

In some forms, the sulfenylated peptide formed from the reaction between the peptide and the sulfynyl reagent in step (i) can have any one of the following structures:

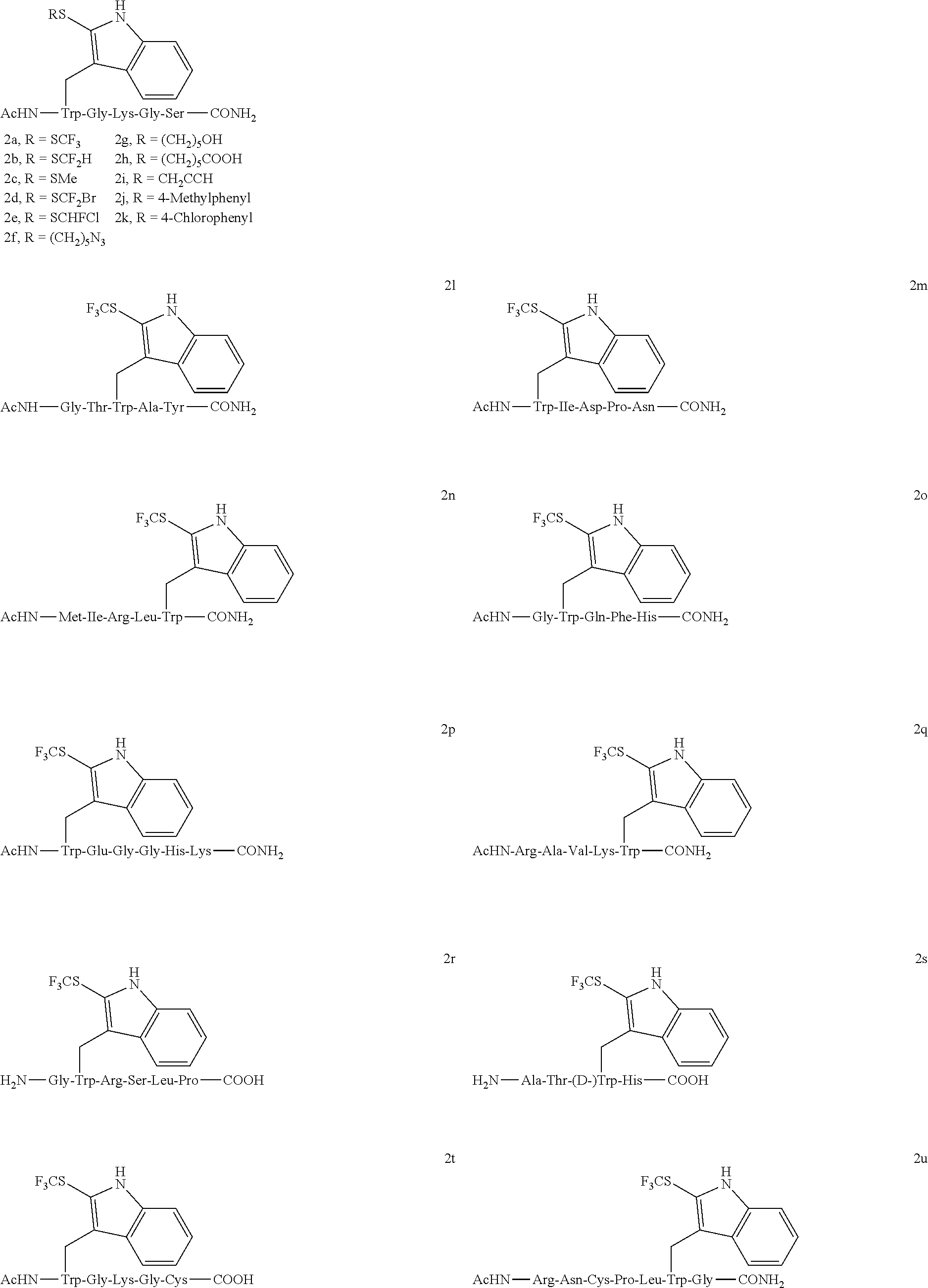

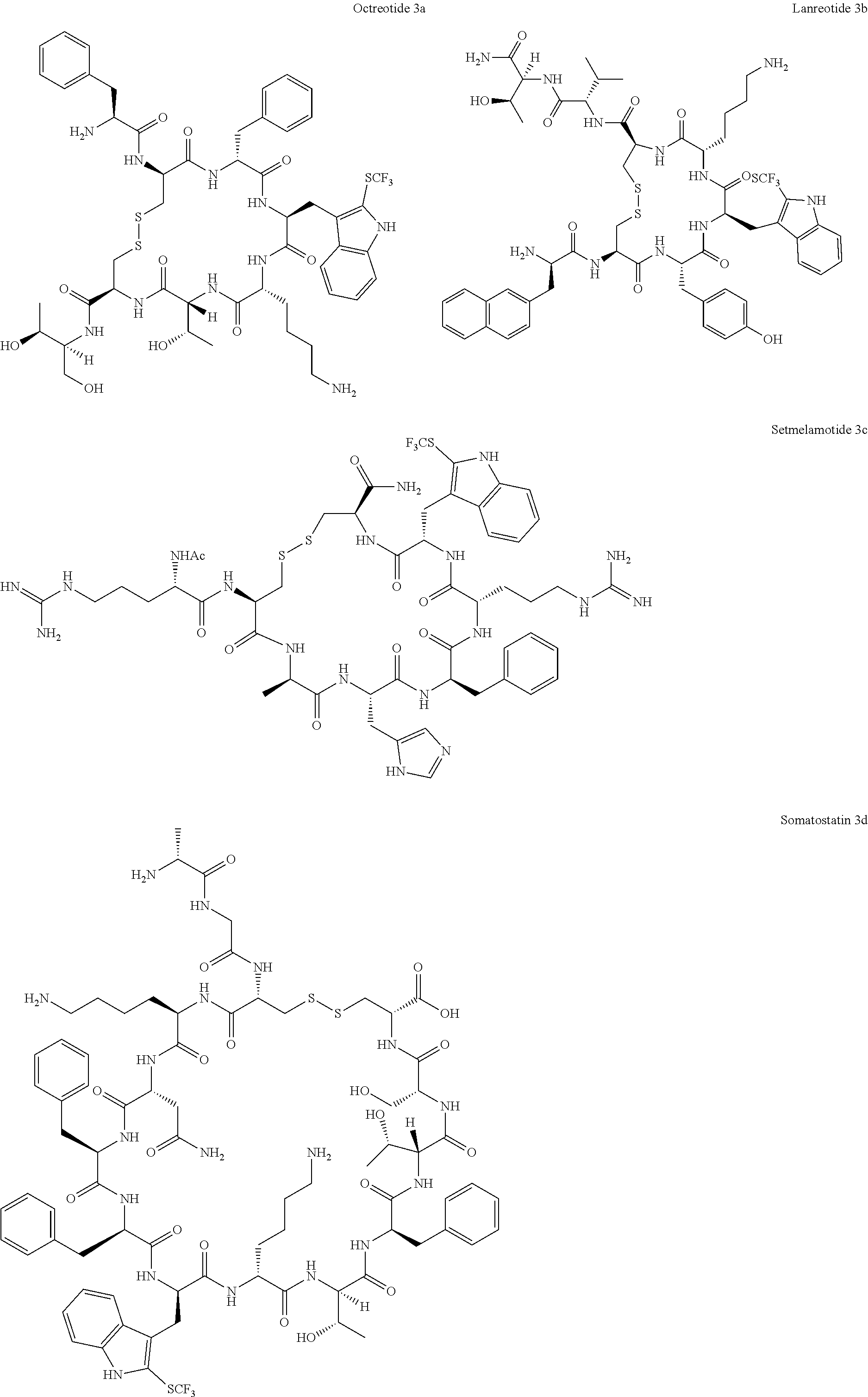

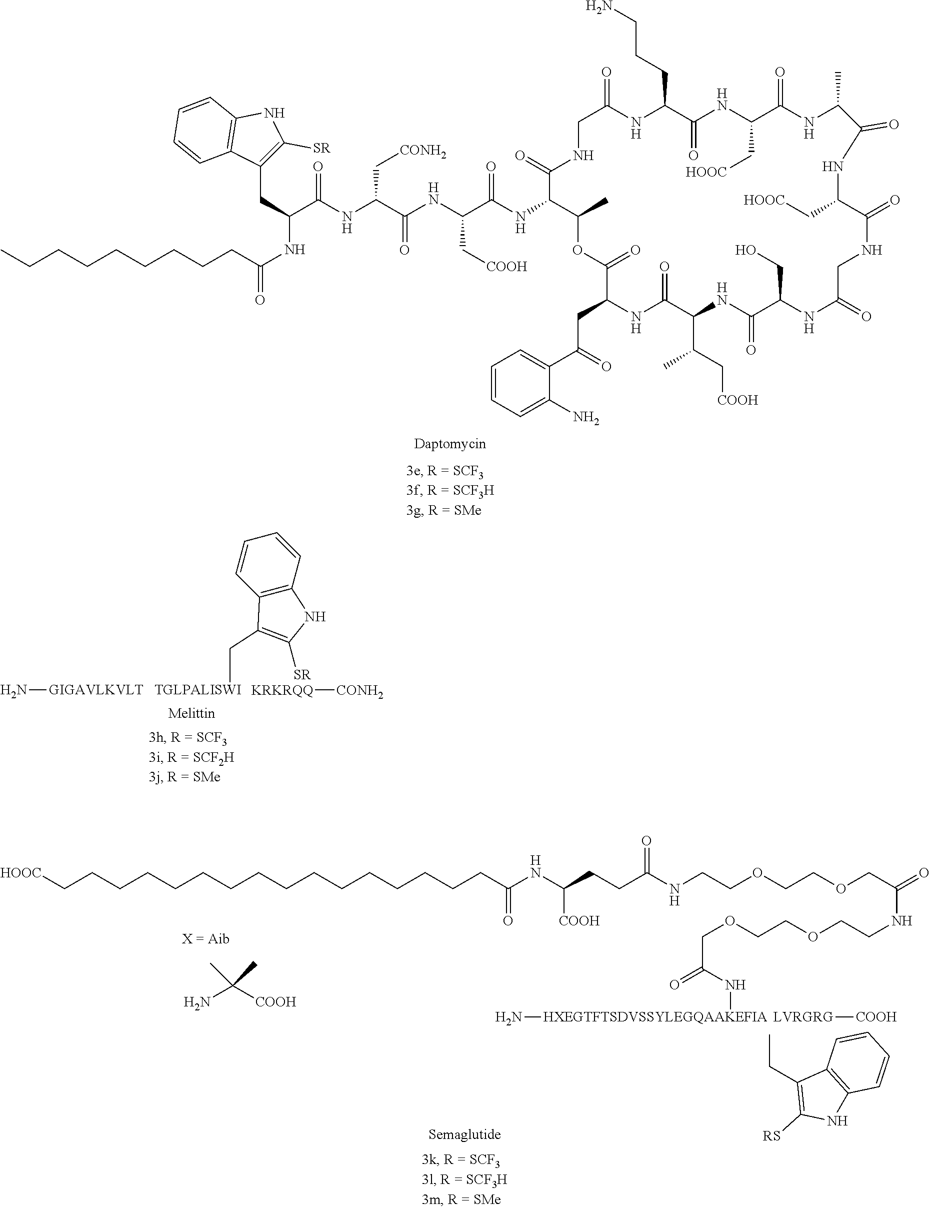

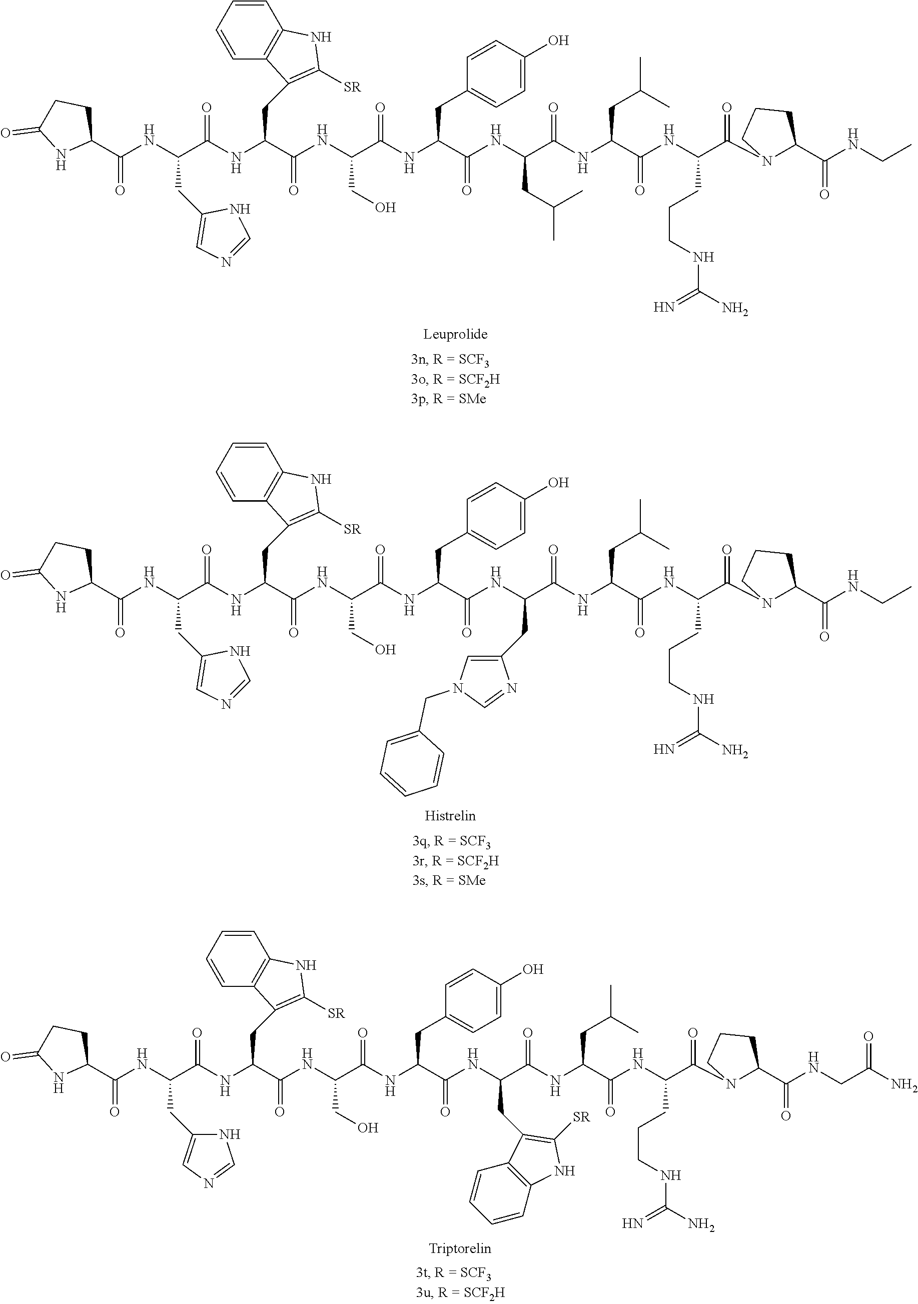

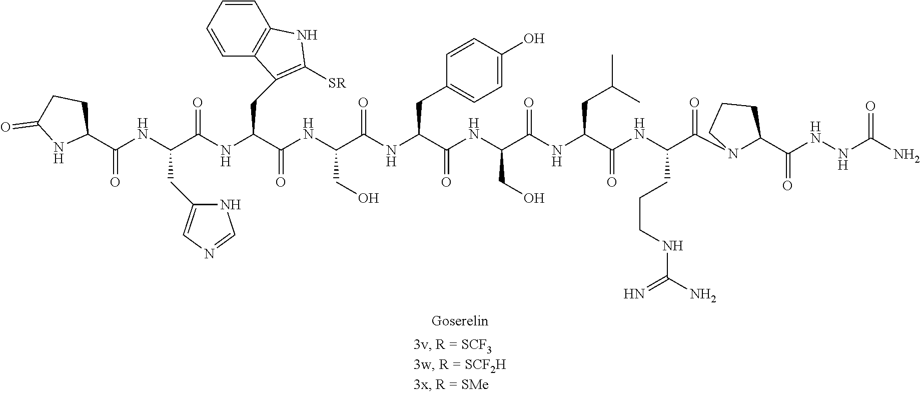

Optionally, the method disclosed herein further includes a step of stirring the reaction mixture during step (i), drying the product to remove the solvent, purifying the product, and/or lyophilizing the sulfenylated peptide. These optional steps can be performed using any suitable techniques known in the art. For example, the purification step can be performed using precipitation, extraction, washing, separation using column chromatography (such as by using a silica gel column), or filtration, or a combination thereof. For example, the product formed in step (i) is purified by precipitating the product using a precipitation solvent (such as ethyl ether) to obtain a precipitate, dissolving the precipitate in a separation solvent (such as a mixture of ACN and water) to obtain a solution, and subjecting the solution to high-performance liquid chromatograph to separate the sulfenylated peptide from impurities.

Generally, the sulfenylated peptide obtained in step (i) has a yield of at least 50%, at least 55%, at least 60%, at least 70%, or at least 80%. The yield of the sulfenylated peptide can be calculated using the formula: (the mole of the sulfenylated peptide)/(the mole of the peptide)*100%. The yield may be determined either before or after purification of the product.

An exemplary reaction scheme of the disclosed late-stage modification methods showing specific reaction conditions is shown in Scheme 1 in the Example below.

The disclosed methods and compounds can be further understood through the following numbered paragraphs.

Paragraph 1. A method of late-stage modification of a peptide comprising:
(i) maintaining a reaction mixture at a sufficient temperature for a sufficient period of time to form a product,
wherein the reaction mixture comprises the peptide comprising one or more tryptophan residues, a sulfynyl reagent, and a solvent,
and wherein the product comprises a sulfenylated peptide, and the sulfenylation is on at least one of the one or more tryptophan residues of the peptide.

Paragraph 2. The method of paragraph 1, wherein the peptide is linear or cyclic (including monocyclic, bicyclic, etc.) Paragraph 3. The method of paragraph 1 or 2, wherein the peptide is a random peptide or a peptide drug.

Paragraph 4. The method of any one of paragraphs 1-3, wherein the peptide comprises natural amino acids.

Paragraph 5. The method of any one of paragraphs 1-4, wherein the sulfynyl reagent is a thiosulfonate reagent having the structure of:

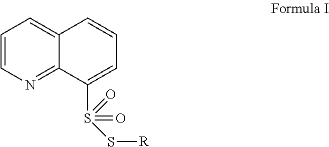

wherein R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, a hydroxyl, an azido, an alkyl, an alkynyl, an alkoxy, a carbonyl (e.g., carboxyl), a nitro, or a cyano.

Paragraph 6. The method of any one of paragraphs 1-5, wherein the solvent is trifluoroacetic acid.

Paragraph 7. The method of paragraph 6, wherein trifluoroacetic acid acts as catalyst and/or is the only solvent in the reaction mixture.

Paragraph 8. The method of any one of paragraphs 1-7, wherein the reaction mixture is maintained at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

Paragraph 9. The method of any one of paragraphs 1-8, wherein the peptide and the sulfynyl reagent have a molar ratio (peptide:sulfynyl reagent) ranging from 0.1 to 1, such as about 0.2.

Paragraph 10. The method of any one of paragraphs 1-9, wherein the peptide has a structure of:

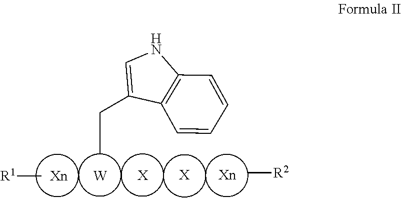

and the sulfenylated peptide has a structure of:

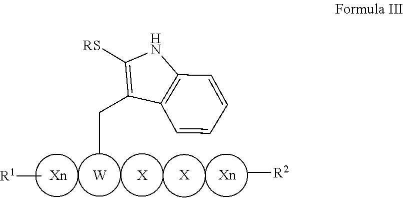

wherein each n is independently 0 or a positive integer (such as an integer from 1 to 50); R₁ is an amino group (such as —NH₂ or NHAc); R₂ is a carbonyl group (such as —COOH) or an amido group (such as —CONH₂); each X is independently a natural amino acid residue (including L- and D-configurations); R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, hydroxyl, an azido, an alkyl, an alkynyl, an alkoxy, a carbonyl (e.g., carboxyl), a nitro, or a cyano; and

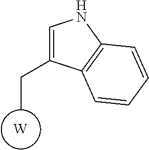

represents a L- or D-tryptophan residue.

Paragraph 11. The method of paragraph 10, wherein X is not a tryptophan residue.

Paragraph 12. The method of any one of paragraphs 1-9, wherein the peptide is a peptide drug, and the sulfenylated peptide is the peptide drug comprising one or more sulfenylated tryptophan residues represented by

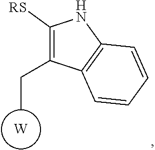

wherein R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, a hydroxyl, an azido, an alkyl, an alkynyl, an alkoxy, a carbonyl (e.g., carboxyl), a nitro, or a cyano.

Paragraph 13. The method of paragraph 12, wherein the peptide drug is octreotide, lanreotide, setmelanotide, somatostain, semaglutide, leuprolide, histrelin, triptorelin, goserelin, melltin, or daptomycin.

Paragraph 14. The method of any one of paragraphs 10-13, wherein R is —CF₃, —CF₂H, —CF₂Br, —CHFCl, —CF₂-alkyl, —CHF-alkyl, a substituted or unsubstituted alkyl (e.g., a substituted or unsubstituted C1-C6 alkyl, a substituted or unsubstituted C1-C5 alkyl, a substituted or unsubstituted C1-C4 alkyl, or a substituted or unsubstituted C1-C3 alkyl), a substituted or unsubstituted alkynyl (e.g., —CH₂CCH), or a substituted or unsubstituted aryl (e.g., a substituted or unsubstituted phenyl), and wherein the substituent(s), when present, is a halogen, a hydroxyl, a carboxyl, an azido, an alkyl (e.g., an unsubstituted C1-C6 alkyl, for example, methyl, ethyl, propyl, or butyl), an alkyne, or an alkoxy.

Paragraph 15. The method of any one of paragraphs 1-14, wherein the sulfenylated peptide has any one of the following structures:

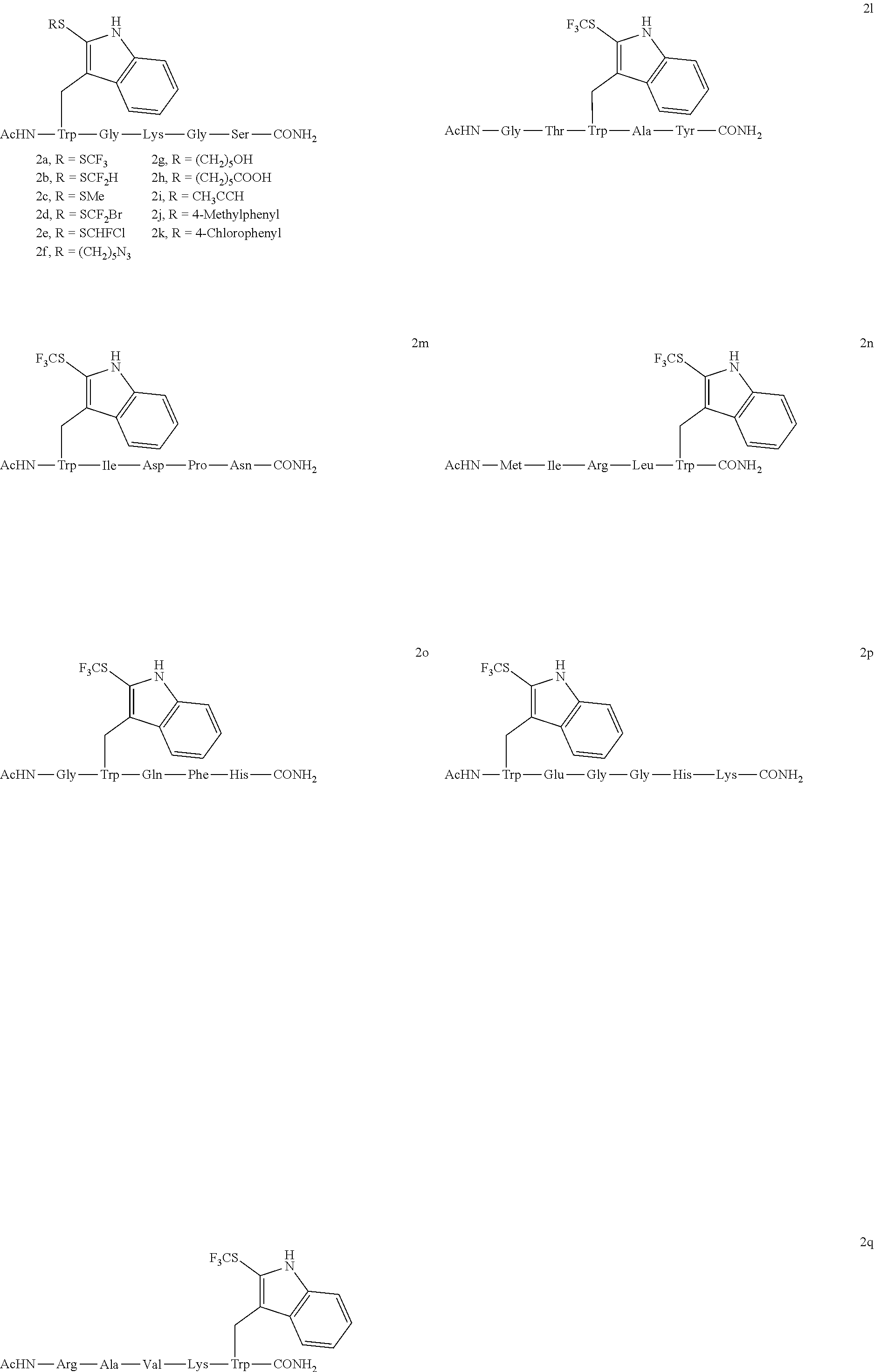

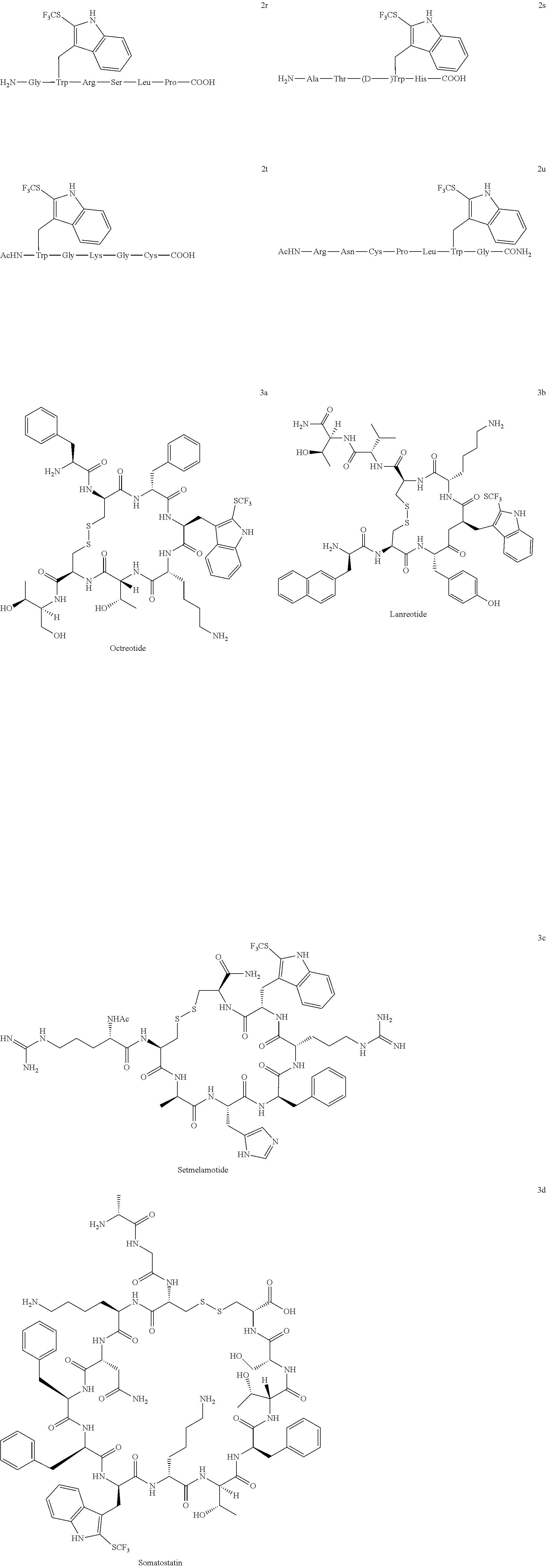

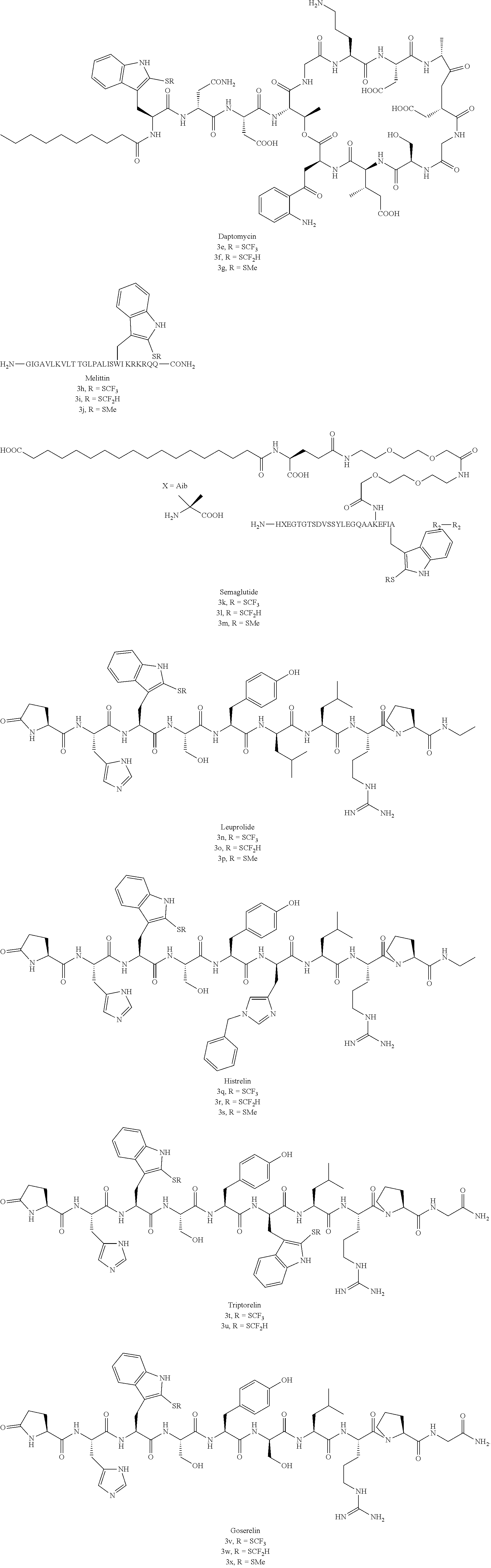

Paragraph 16. The method of any one of paragraphs 1-15, further comprising stirring the reaction mixture during step (i), drying the product to remove the solvent, purifying the product, and/or lyophilizing the sulfenylated peptide.

Paragraph 17. The method of paragraph 16, wherein the product is purified by precipitating the product using a precipitation solvent (such as ethyl ether) to obtain a precipitate, dissolving the precipitate in a separation solvent (such as a mixture of ACN and water) to obtain a solution, and subjecting the solution to high-performance liquid chromatograph to separate the sulfenylated peptide from impurities.

Paragraph 18. The method of any one of paragraphs 1-17, wherein the sulfenylated peptide has a yield of at least 50%, at least 55%, at least 60%, at least 70%, or at least 80%.

Paragraph 19. A sulfenylated peptide comprising one or more sulfenylated tryptophan residues represented by

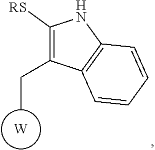

wherein R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, hydroxyl, an azido, an alkyl, an alkynyl, an alkoxy, a carbonyl (e.g., carboxyl), a nitro, or a cyano.

Paragraph 20. The sulfenylated peptide of paragraph 19, wherein the peptide is a linear or cyclic peptide.

Paragraph 21. The sulfenylated peptide of paragraph 19 or 20, wherein the peptide is a random peptide or a peptide drug.

Paragraph 22. The sulfenylated peptide of any one of paragraphs 19-21, having a structure of:

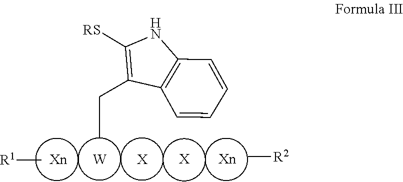

wherein each n is independently 0 or a positive integer (such as an integer from 1 to 50); R₁ is an amino group (such as —NH₂ or NHAc); R₂ is a carbonyl group (such as —COOH) or an amido group (such as —CONH₂); each X is independently a natural amino acid residue (including L- and D-configurations); and

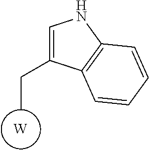

represents a L- or D-tryptophan residue.

Paragraph 23. The sulfenylated peptide of paragraph 22, wherein X is not a tryptophan residue.

Paragraph 24. The sulfenylated peptide of any one of paragraphs 19-21, wherein the peptide is a peptide drug, optionally wherein the peptide drug is octreotide, lanreotide, setmelanotide, somatostain, semaglutide, leuprolide, histrelin, triptorelin, goserelin, melltin, or daptomycin.

Paragraph 25. The sulfenylated peptide of any one of paragraphs 19-24, wherein R is —CF₃, —CF₂H, —CF₂Br, —CHFCl, —CF₂-alkyl, —CHF-alkyl, a substituted or unsubstituted alkyl (e.g., a substituted or unsubstituted C1-C6 alkyl, a substituted or unsubstituted C1-C5 alkyl, a substituted or unsubstituted C1-C4 alkyl, or a substituted or unsubstituted C1-C3 alkyl), a substituted or unsubstituted alkynyl (e.g., —CH₂CCH), or a substituted or unsubstituted aryl (e.g., a substituted or unsubstituted phenyl), and wherein the substituent(s), when present, is a halogen, a hydroxyl, a carboxyl, an azido, an alkyl (e.g., an unsubstituted C1-C6 alkyl, for example, methyl, ethyl, propyl, or butyl), an alkynyl, or an alkoxy.

Paragraph 26. The sulfenylated peptide of any one of paragraphs 19-25, having any one of the following structures:

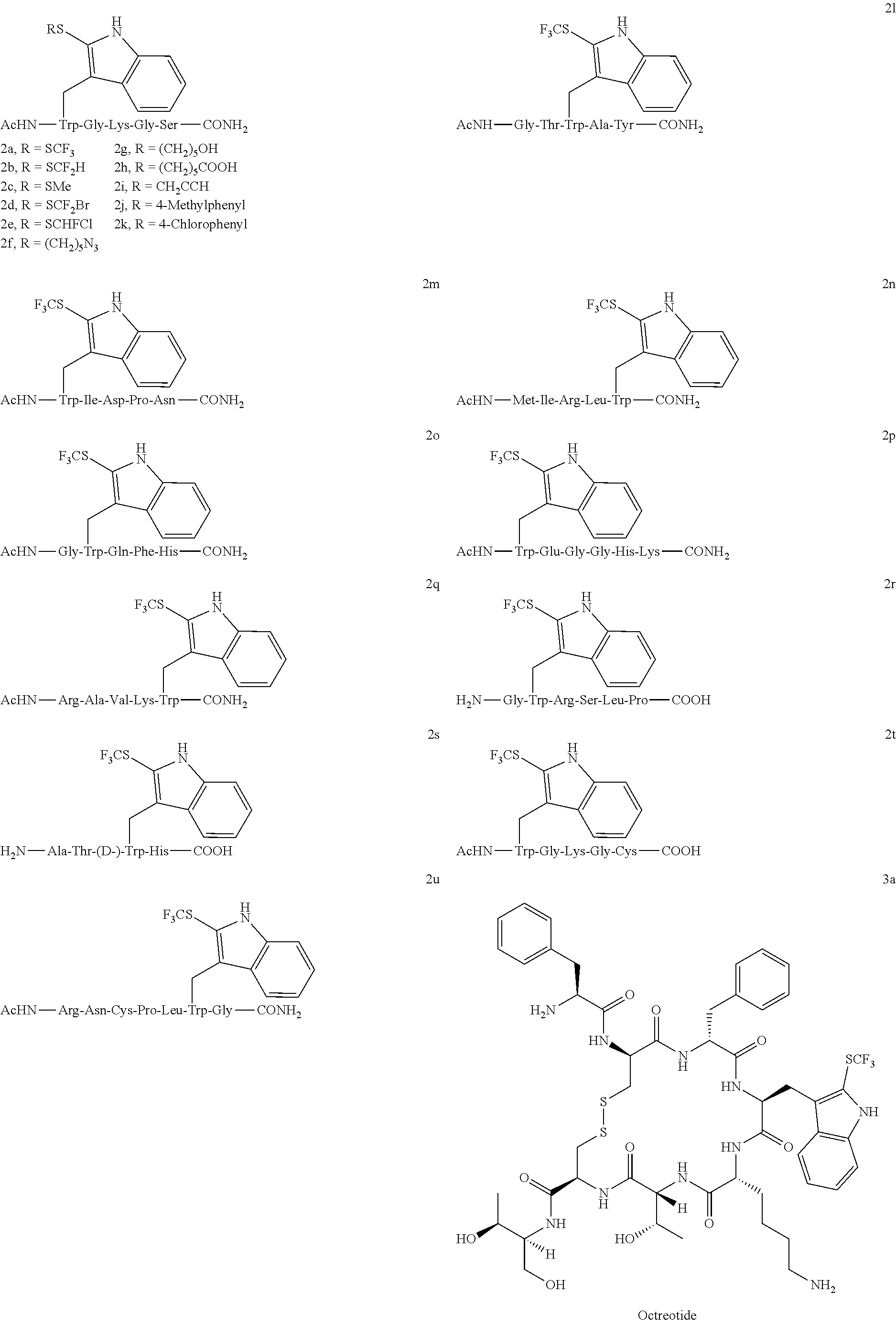

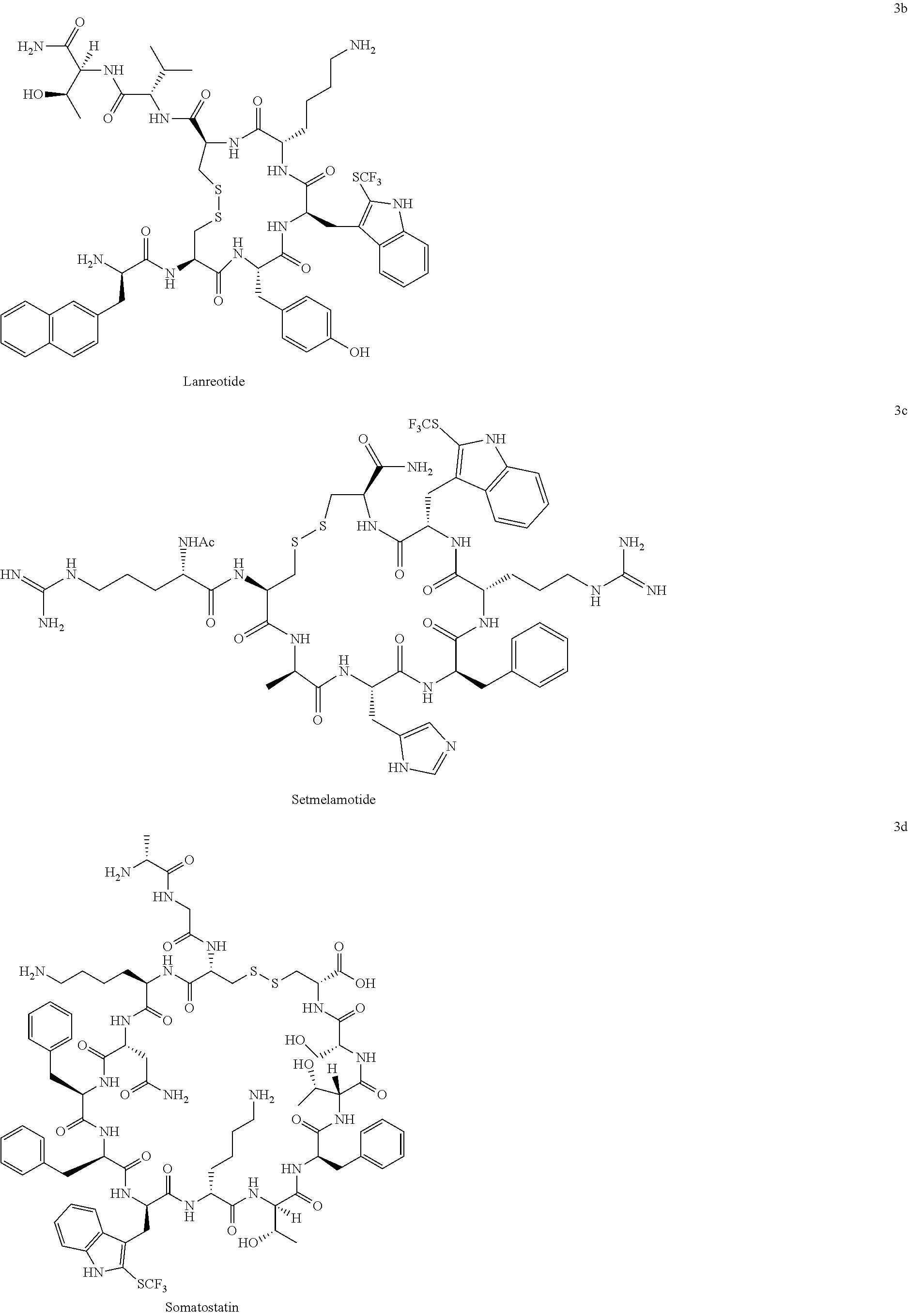

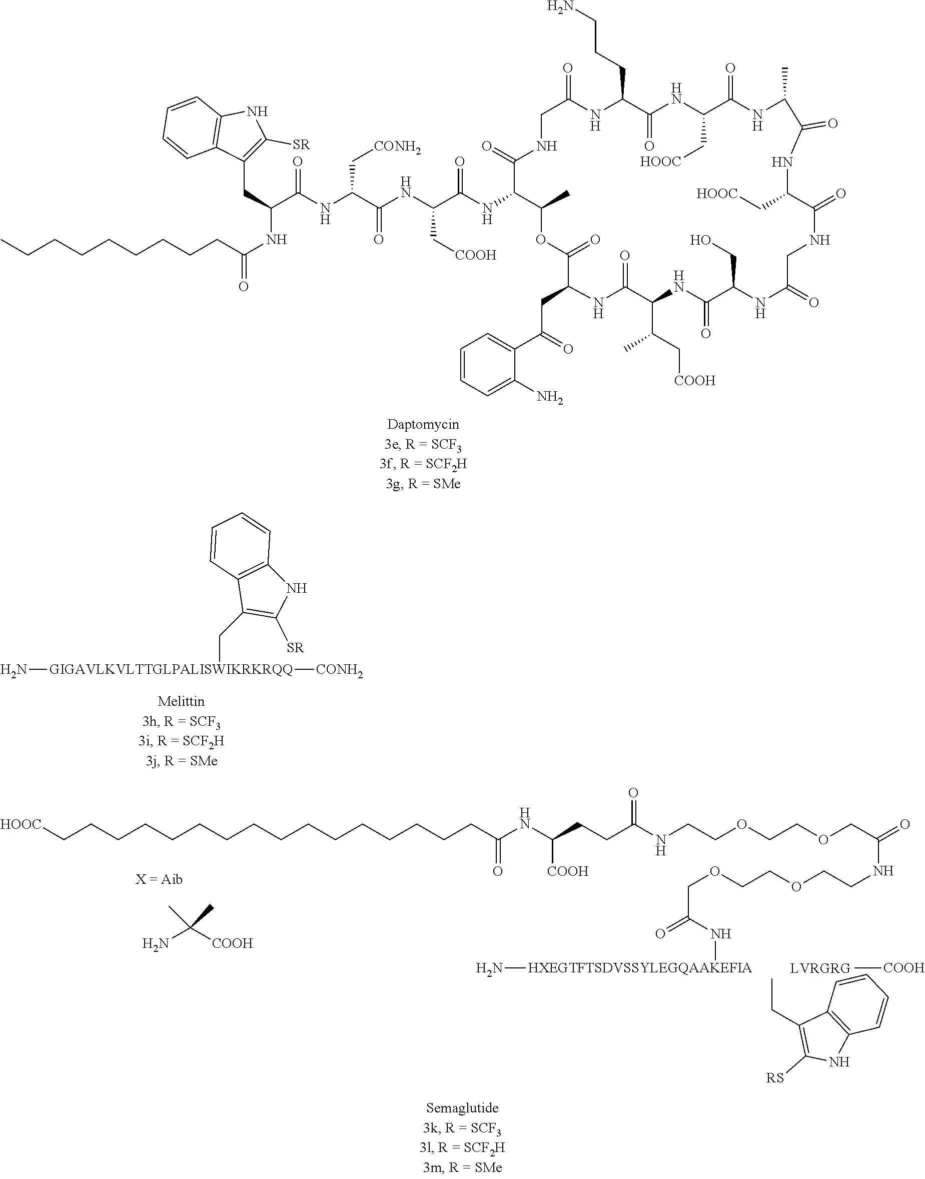

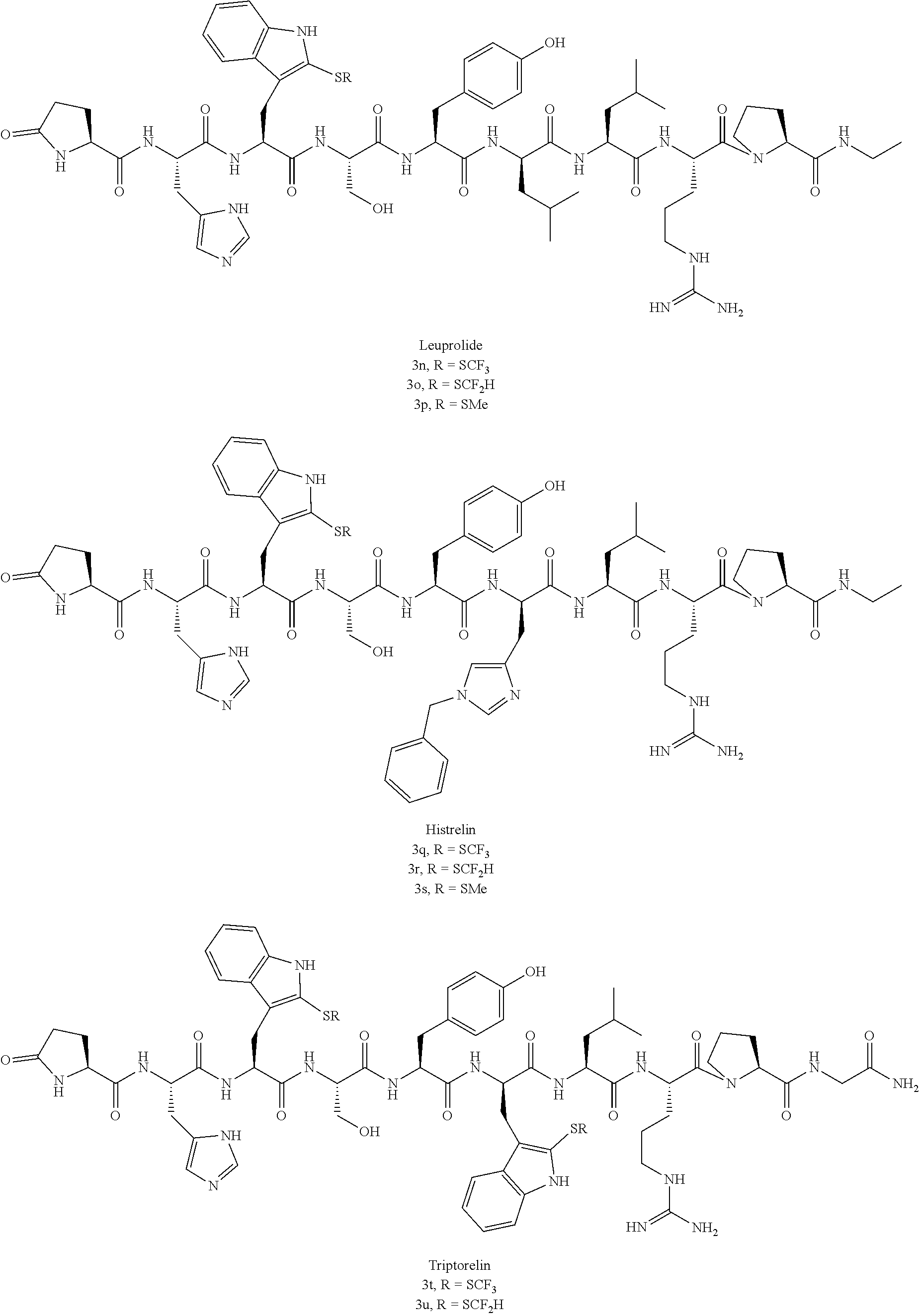

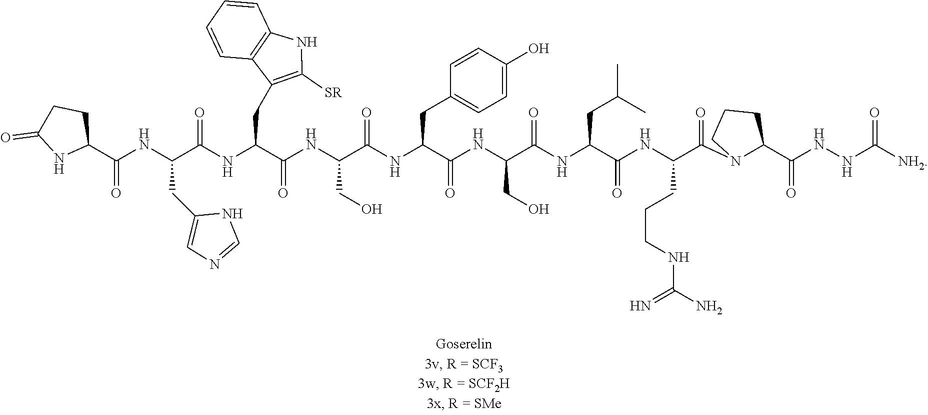

The detailed conditions of exemplary modification methods and exemplary sulfenylated peptides are described below in the following non-limiting examples.

### EXAMPLES

### Example 1. Precise Late-Stage Modification on Tryptophan of Native Peptides Using Sulfynyl Reagents

### Materials and Methods

The general protocol of the precise late-stage modification on tryptophan of native peptides is shown in Scheme 1,

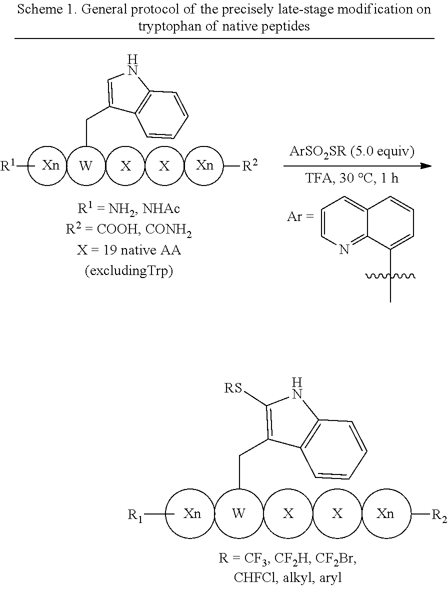

where the native amino acid X can be L- or D-amino acid; R is optionally substituted with one or more functional groups, such as halogen, azide, alkyne, and alkoxy; and

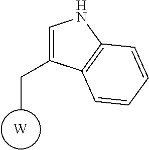

represents L- or D-tryptophan residue.

Purified random peptide sequences from Solid-phase peptide synthesis (SPPS) and thiosulfonate (ArSO₂SR) reagents were dissolved in trifluoroacetic acid (TFA) with a final concentration of 10 mM and 50 mM respectively. The reaction was stirred at 30° C. for 1 h. After finishing the reaction, the solvent was blown away by compressed air.

The crude product was precipitated by ethyl ether. The residue was dissolved in the mix solvent of acetonitrile (ACN) and water (H₂O) then subjected to preparative HPLC purification. After lyophilization could afforded corresponding product. Here, the detail synthetic procedures of late-stage peptide modification with the thiosulfonate reagents described as follow as examples. Examples of peptide sequences modified using the general protocol as shown in Scheme 1 and described above are shown in Scheme 2.

### Scheme 2 Representative Examples of Late-Stage Modification of Random Peptide

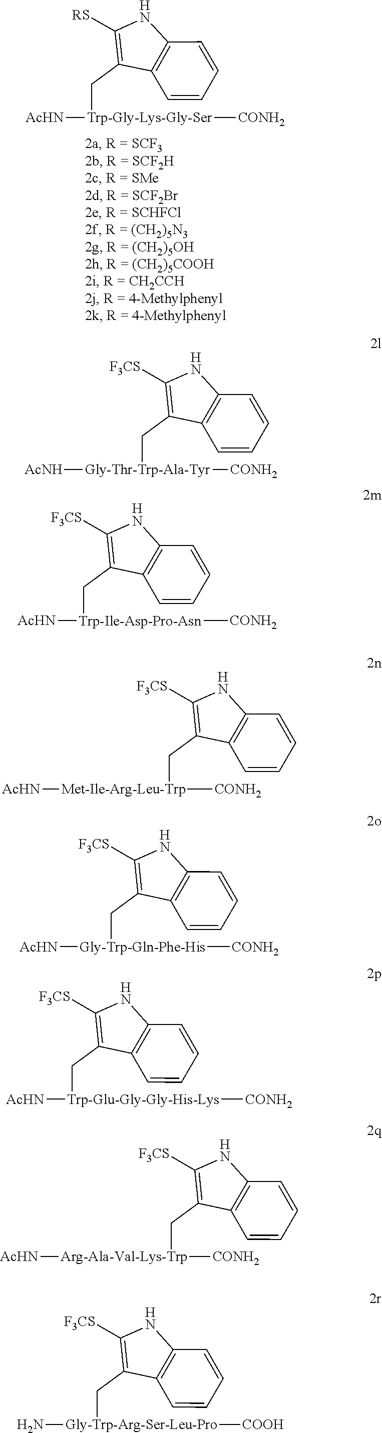

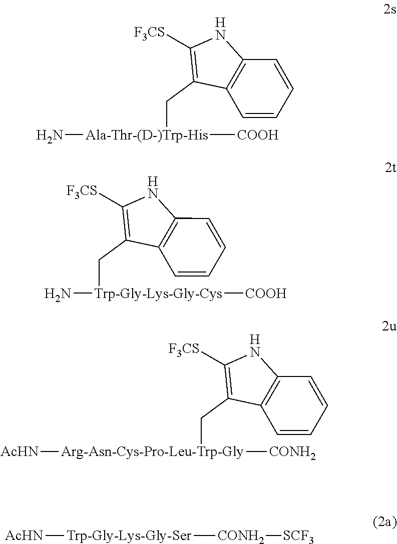

Purified AcHN-Trp-Gly-Lys-Gly-Ser-CONH₂ (I a) after SPPS (5.7 mg, 1.0 equiv) and thiosulfonate reagent (ArSO₂SCF₃) (14.6 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 10% ACN/H₂O and subjected to preparative HPLC purification (10-60%, over 35 min). Lyophilization afforded 2a as a white powder (5.2 mg, 59%).

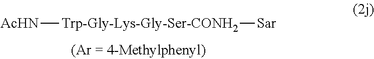

Purified AcHN-Trp-Gly-Lys-Gly-Ser-CONH₂ (1a) after SPPS (5.7 mg, 1.0 equiv) and thiosulfonate reagent (ArSO₂SAr, Ar=4-Methylphenyl) (15.7 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 20% ACN/H₂O and subjected to preparative HPLC purification (20-70%, over 35 min). Lyophilization afforded 2j as a white powder (4.7 mg, 67%).

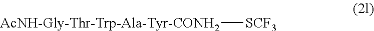

Purified AcNH-Gly-Thr-Trp-Ala-Tyr-CONH₂ (1b) after SPPS (6.3 mg, 1.0 equiv) and thiosulfonate reagent (ArSO₂SCF₃) (14.6 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 15% ACN/H₂O and subjected to preparative HPLC purification (15-60%, over 35 min). Lyophilization afforded 21 as a white powder (5.7 mg, 77%).

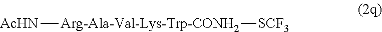

Purified AcHN-Arg-Ala-Val-Lys-Trp-CONH₂ (1g) after SPPS (6.6 mg, 1.0 equiv) and thiosulfonate reagent (ArSO₂SCF₃) (14.6 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 15% ACN/120 and subjected to preparative HPLC purification (15-60%, over 35 min). Lyophilization afforded 2q as a white powder (6.6 mg, 82%).

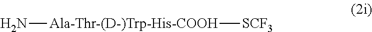

Purified H₂N-Ala-Thr-(D-)Trp-His-COOH (1i) after SPPS (6.2 mg, 1.0 equiv) and thiosulfonate reagent (ArSO₂SCF₃) (14.6 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 10% ACN/H₂O and subjected to preparative HPLC purification (10-60%, over 35 min). Lyophilization afforded 2i as a white powder (5.4 mg, 74%).

The general method of late-stage modification of peptide drugs and biomolecules is shown in Scheme 3.

Specifically, commercial or purified peptide drugs and biomolecules from Solid-phase peptide synthesis (SPPS) and thiosulfonate (ArSO₂SR) reagent were dissolved in trifluoroacetic acid (TFA) with a final concentration of 10 mM and 50 mM respectively. The reaction was stirred at 30° C. for 1 h or 2 hrs. After completion of the reaction, the solvent was blown away by compressed air. The crude peptide product was precipitated by ethyl ether. The residue was dissolved in the mix solvent of acetonitrile (ACN) and water (H₂O) then subjected to preparative HPLC purification. After lyophilization could afforded corresponding product. The detail synthetic procedures of late-stage peptide modification with cyclic ketones were described as follow as examples.

### Scheme 3 Representative Examples of Late-Stage Modification of Peptide Drugs and Biomolecules

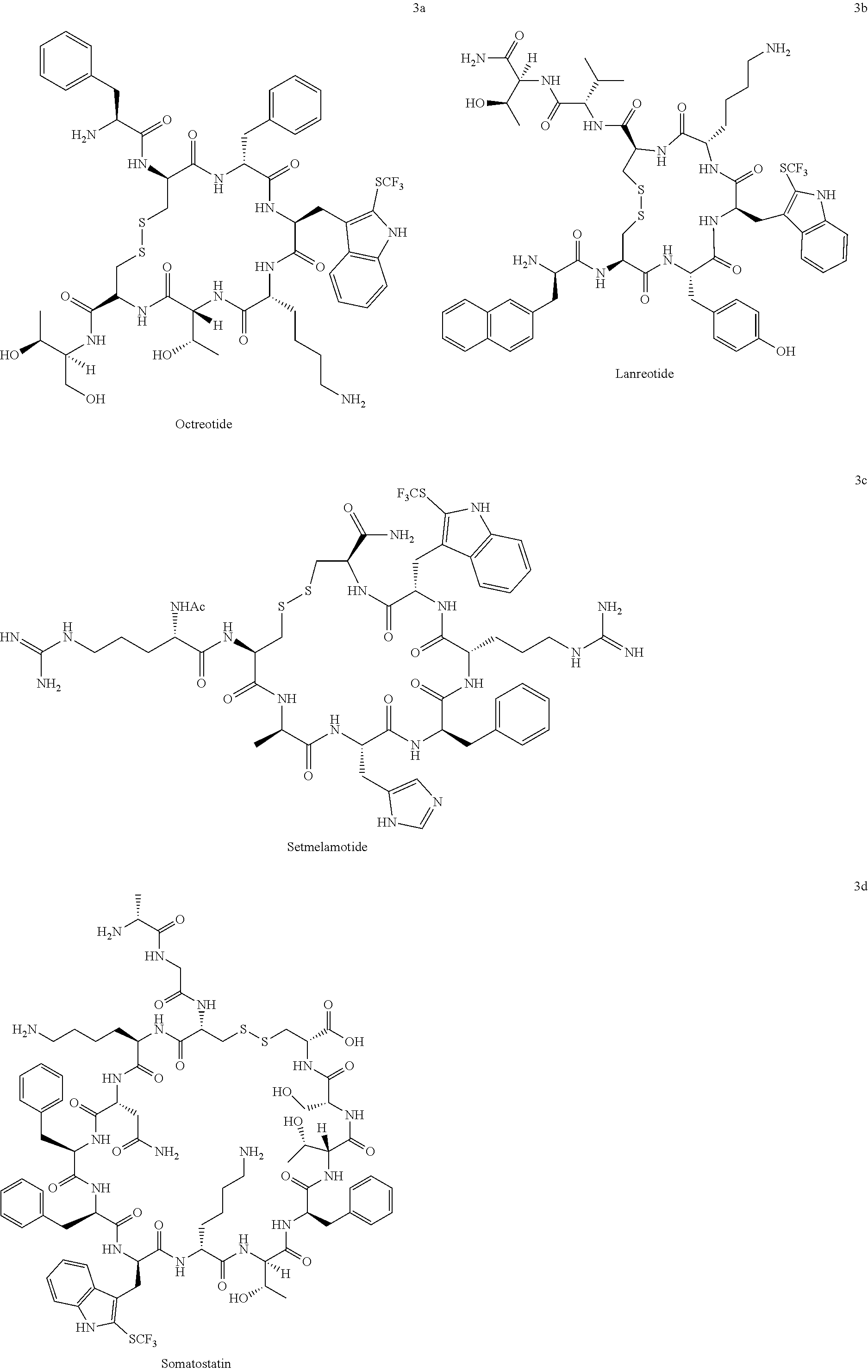

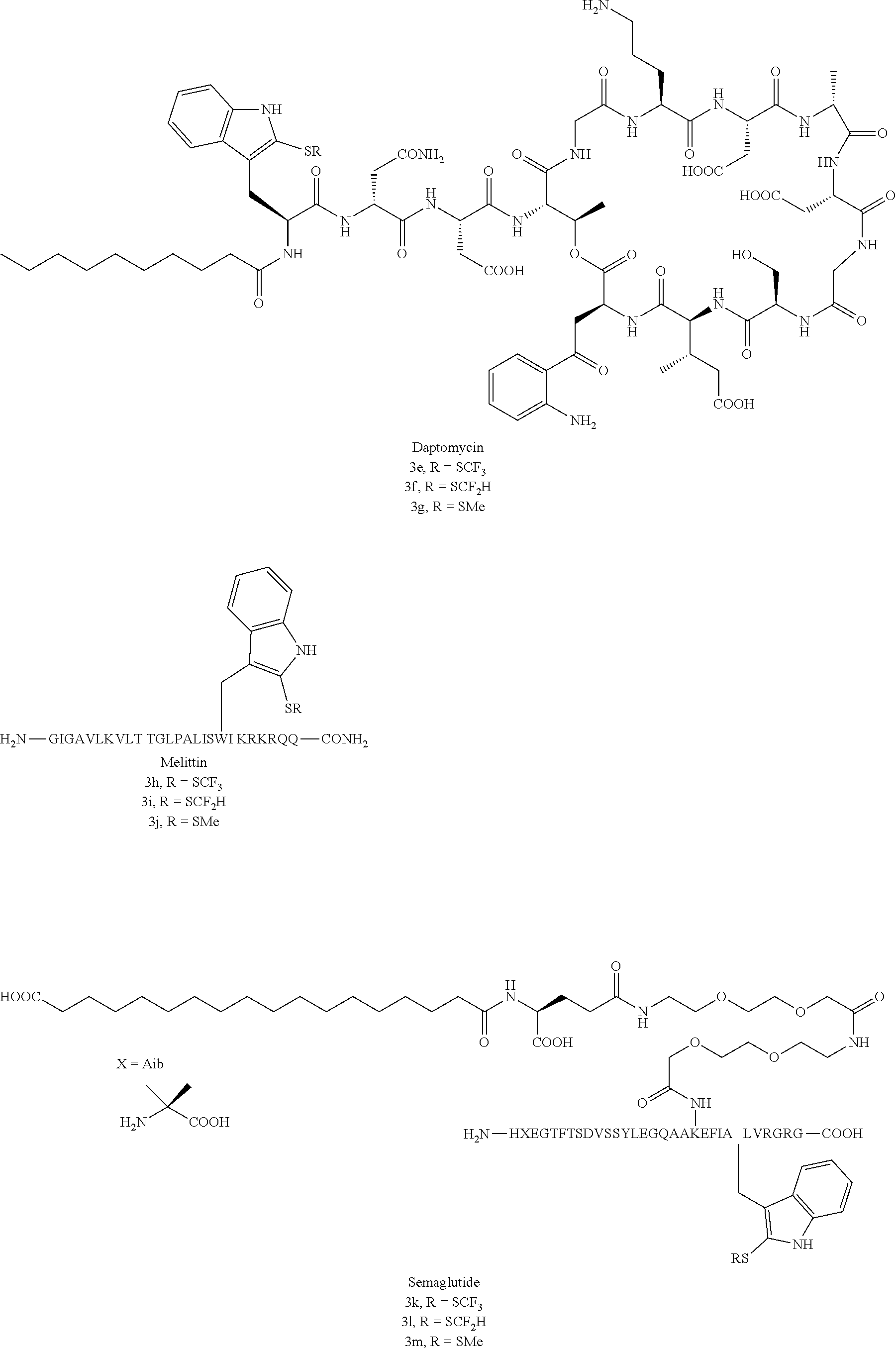

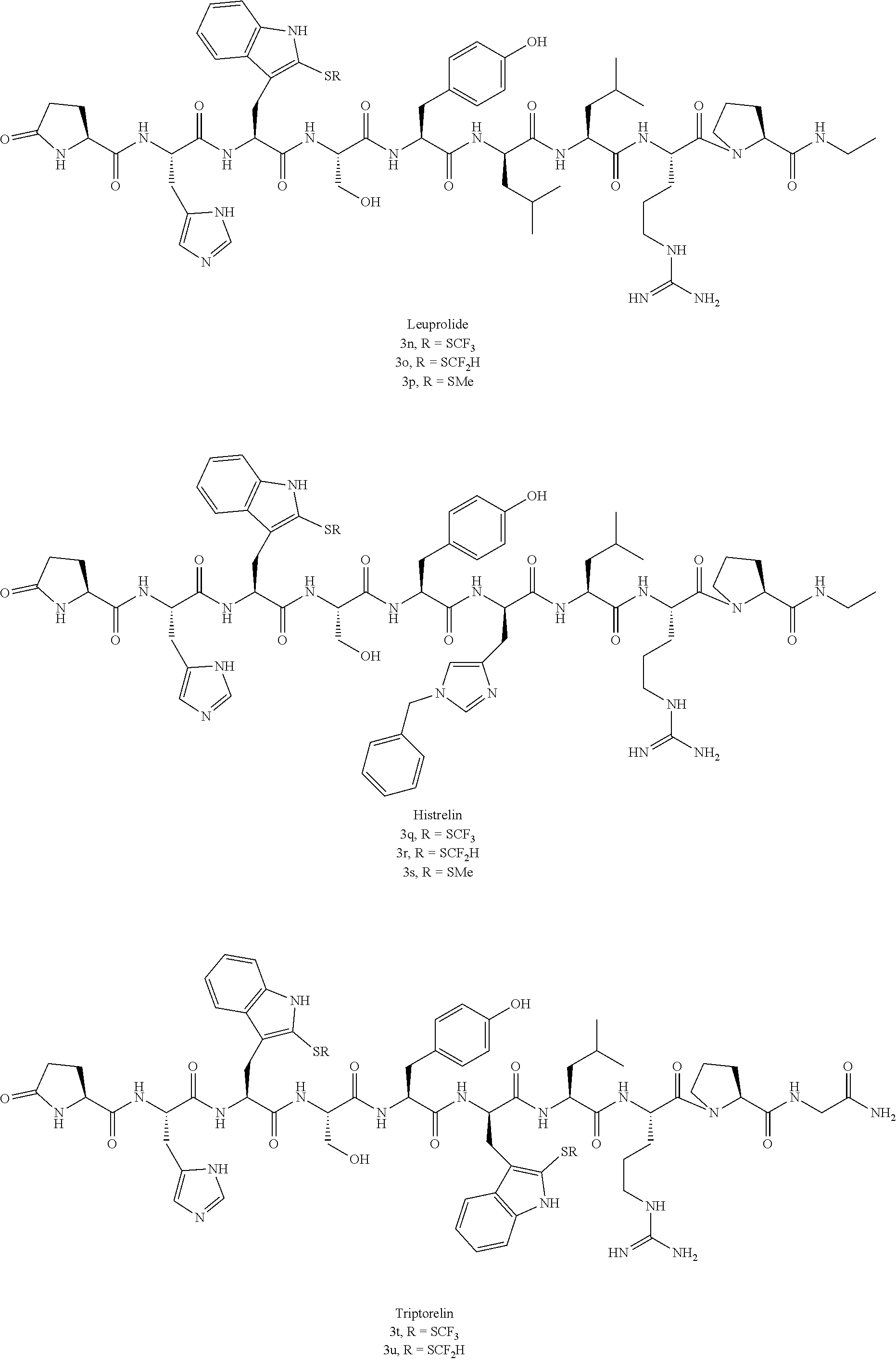

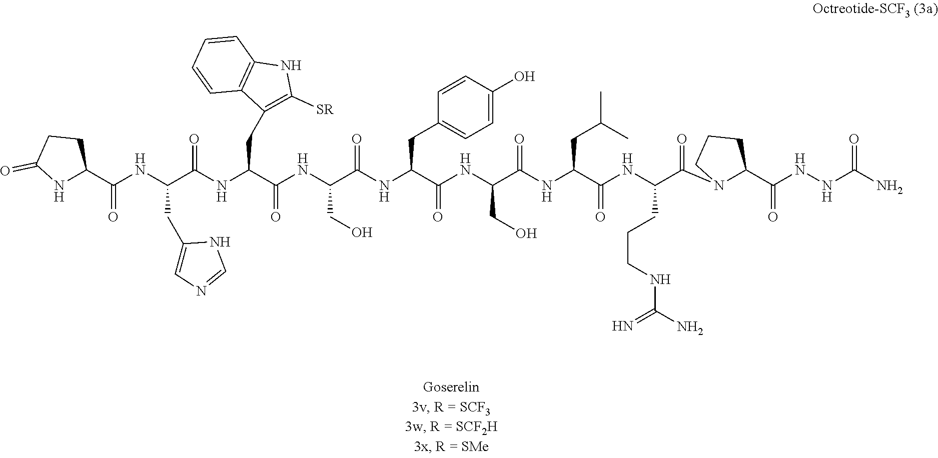

Octrotide (3.8 mg, 1.0 equiv) and thiosulfonate reagent (ArSO₂SCF₃) (7.3 mg, 5.0 equiv) were dissolved in trifluoroacetic acid (TFA) with a final concentration of 10 mM and 50 mM respectively. The reaction was stirred at 30° C. for 2 h. After finishing the reaction, the solvent was blown away by compressed air. The crude peptide product was precipitated by ethyl ether. The residue was dissolved in 10 mL 20% ACN/H₂O and subjected to preparative HPLC purification (20-60%, over 35 min). Lyophilization afforded 3a as a white powder (3.8 mg, 68%).

### Somatostatin-SCF₃ (3d)

Somatostatin (8.1 mg, 1.0 equiv) and thiosulfonate reagent (ArSO₂SCF₃) (7.3 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 20% ACN/H₂O and subjected to preparative HPLC purification (20-60%, over 35 min). Lyophilization afforded 3d as a white powder (5.9 mg, 68%).

### Daptomycin-SCF₃ (3e)

Daptomycin (16.2 mg, 1.0 equiv) and thiosulfonate reagent (ArSO₂SCF₃) (14.6 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 30% ACN/H₂O and subjected to preparative HPLC purification (30-80%, over 35 min). Lyophilization afforded 3e as a white powder (13.2 mg, 77%).

### Daptomycin-SCF₂H (3f)

Daptomycin (6.8 mg, 1.0 equiv) and thiosulfonate reagent (ArSO₂SCF₂H) (6.9 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 30% ACN/H₂O and subjected to preparative HPLC purification (30-80%, over 35 min). Lyophilization afforded 3f as a white powder (6.8 mg, 80%).

### Daptomycin-SMe (3g)

Daptomycin (8.1 mg, 1.0 equiv) and thiosulfonate reagent (ArSO₂SCF₃) (6.0 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 30% ACN/H₂O and subjected to preparative HPLC purification (30-80%, over 35 min). Lyophilization afforded 3g as a white powder (6.2 mg, 75%).

### Semaglutide-SCF₃ (3k)

Semaglutide (8.2 mg, 1.0 equiv) and thiosulfonate reagent (ArSO₂SCF₃) (2.9 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 40% ACN/H₂O and subjected to preparative HPLC purification (40-80%, over 35 min). Lyophilization afforded 3k as a white powder (6.7 mg, 80%).

### Semaglutide-SCF₂H (31)

Semaglutide (8.2 mg, 1.0 equiv) and thiosulfonate reagent (ArSO₂SCF₂H) (2.7 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 40% ACN/H₂O and subjected to preparative HPLC purification (40-80%, over 35 min). Lyophilization afforded 31 as a white powder (6.4 mg, 76%).

### Semaglutide-SMe (3m)

Semaglutide (8.2 mg, 1.0 equiv) and thiosulfonate reagent (ArSO₂SMe) (2.4 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 40% ACN/H₂O and subjected to preparative HPLC purification (40-80%, over 35 min). Lyophilization afforded 3m as a white powder (6.8 mg, 82%).

### Triptorelin-SCF₃ (3t)

Triptorelin (6.5 mg, 1.0 equiv) and thiosulfonate reagent (ArSO₂SCF₃) (7.3 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 20% ACN/H₂O and subjected to preparative HPLC purification (20-70%, over 35 min). Lyophilization afforded 3t as an off-white powder (4.0 mg, 53%).

### Trptorelin-SCF₃ (3u)

Triptorelin (6.5 mg, 1.0 equiv) and thiosulfonate reagent (ArSO₂SCF₃) (6.9 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude product was precipitated by ethyl ether. The residue was dissolved in 10 mL 20% ACN/H₂O and subjected to preparative HPLC purification (20-70%, over 35 min). Lyophilization afforded 3u as an off-white powder (3.7 mg, 50%).

### Results

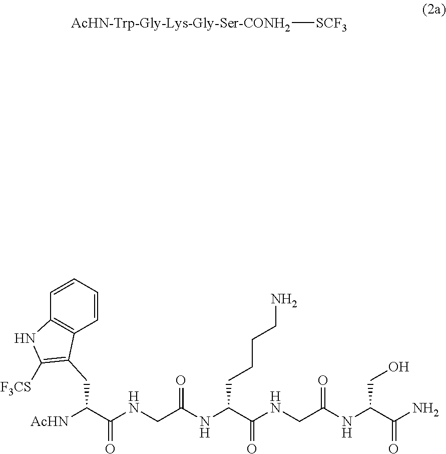

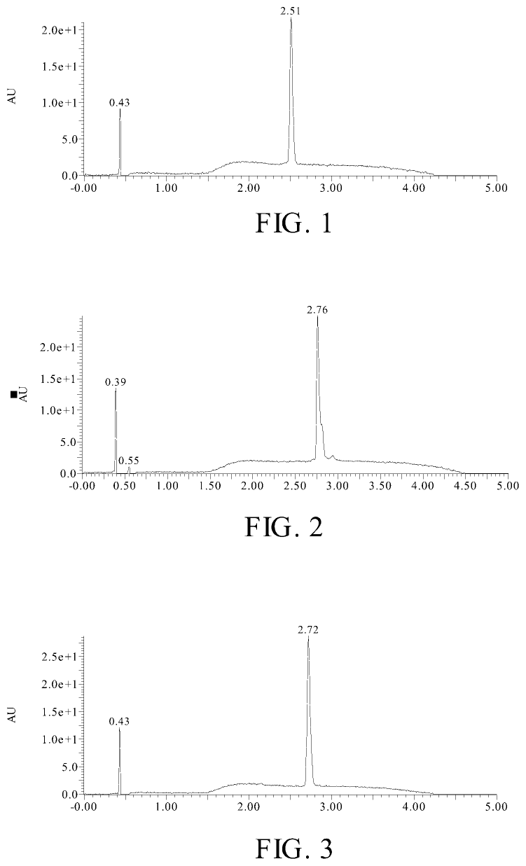

UV (190-400 nm) and MS (250-3000 m/z) traces from UPLC-MS analysis of 2a (gradient 5-95% CH₃CN/H₂O containing 0.1% TFA over 5 min at a flow rate of 0.4 mL/min) are shown in FIG. 1. ESI-MS calcd. for C₂₇H₃₇F₃N₈O₇S: [M+H]⁺ m/z=675.7, found 675.6.

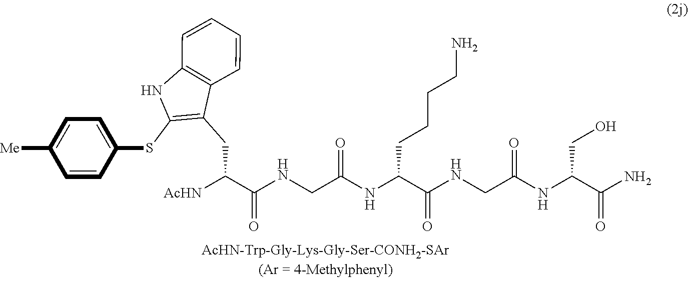

UV (190-400 nm) and MS (250-3000 m/z) traces from UPLC-MS analysis of 2j (gradient 5-95% CH₃CN/H₂O containing 0.1% TFA over 5 min at a flow rate of 0.4 mL/min) are shown in FIG. 2. ESI-MS calcd. for C₃₃H₄₄N₈O₇S: [M+H]⁺ m/z=697.8, found 697.5.

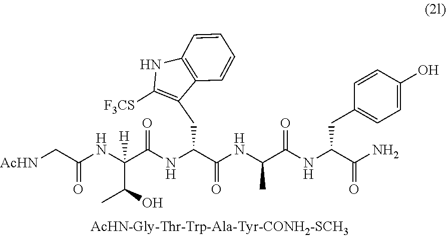

UV (190-400 nm) and MS (250-3000 m/z) traces from UPLC-MS analysis of 21 (gradient 5-95% CH₃CN/H₂O containing 0.1% TFA over 5 min at a flow rate of 0.4 mL/min) are shown in FIG. 3. ESI-MS calcd. for C₃₂H₃₈F₃N₇O₈S: [M+H]⁺ m/z=738.7, found 738.5.

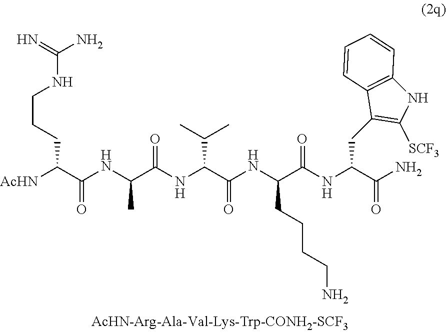

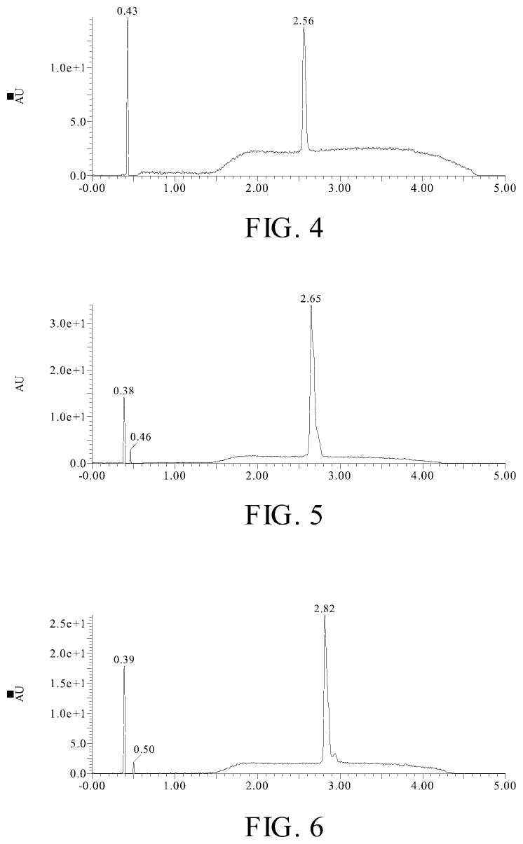

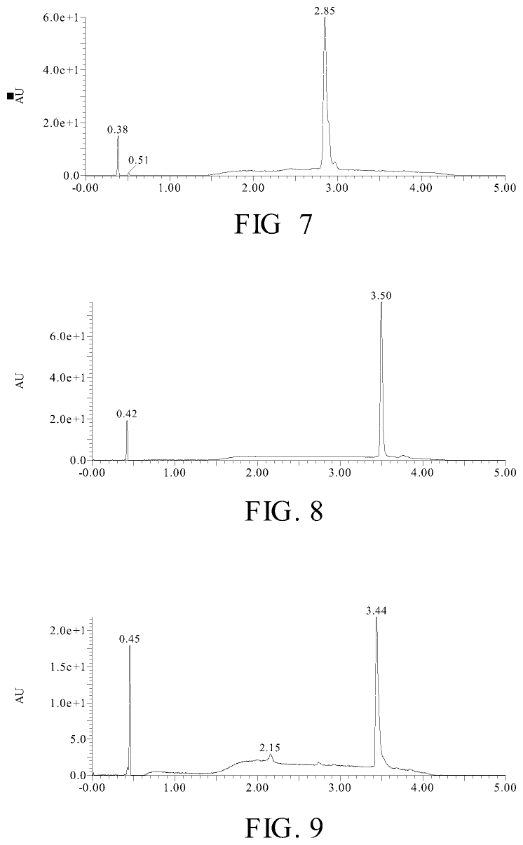

UV (190-400 nm) and MS (250-3000 m/z) traces from UPLC-MS analysis of 2q (gradient 5-95% CH₃CN/H₂O containing 0.1% TFA over 5 min at a flow rate of 0.4 mL/min) are shown in FIG. 4. ESI-MS calcd. for C₃₄H₅₂F₃N₁₁O₆S: [M+H]⁺ m/z=800.9, found 800.3; [M+2H]²⁺ m/z=400.9, found 400.9.

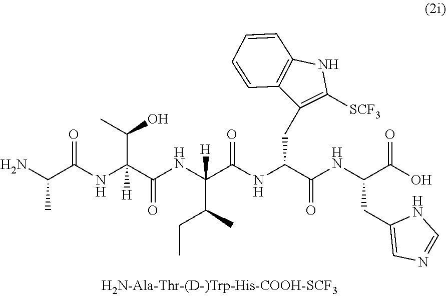

UV (190-400 nm) and MS (250-3000 m/z) traces from UPLC-MS analysis of 21 (gradient 5-95% CH₃CN/H₂O containing 0.1% TFA over 5 min at a flow rate of 0.4 mL/min) are shown in FIG. 5. ESI-MS calcd. for C₃₁H₄₁F₃N₈O₇S: [M+H]⁺ m/z=727.7, found 727.5; [M+2H]²⁺ m/z=364.3, found 364.5.

With the specific and robust late-stage modification protocol, the application scope of this reaction was expanded. Cyclic peptides, such as octreotide, lanreotide, setmelanotide, and somatostain containing disulfide bond, gave smoothly full conversion under suitable conditions. Further, anti-T2DM peptide drug (such as semaglutide), anti-cancer drugs (such as leuprolide, histrelin, triptorelin, and goserelin), and native active biomolecule melltin were also readily derivatized with thiosulfonate (ArSO₂SR) reagent to generate corresponding products in good yields.

### Octreotide-SCF₃ (3a)

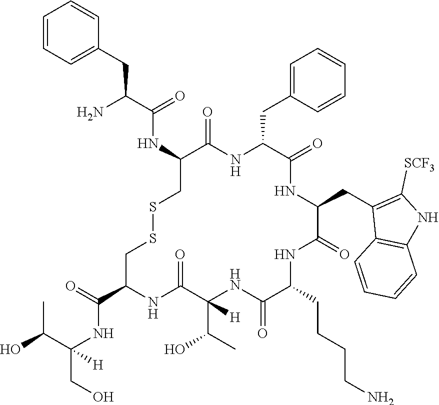

UV (190-400 nm) and MS (250-3000 m/z) traces from UPLC-MS analysis of 3a (gradient 5-95% CH₃CN/H₂O containing 0.1% TFA over 5 min at a flow rate of 0.4 mL/min) are shown in FIG. 6. ESI-MS calcd. for C₅₀H₆₅F₃N₁₀O₁₀S₃: [M+H]⁺ m/z=1120.3, found 1119.8; [M+2H]²⁺ m/z=560.6, found 560.6.

### Somatostatin-SCF₃ (3d)

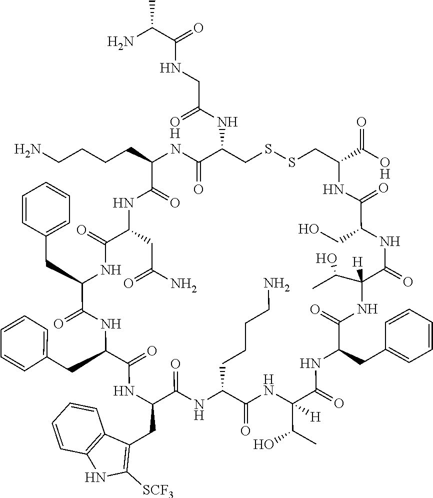

Somatostatin (8.1 mg, 1.0 equiv) and thiosulfonate reagent (ArSO₂SCF₃) (7.3 mg, 5.0 equiv) were dissolved in trifluoroacetic acid (TFA) with a final concentration of 10 mM and 50 mM respectively. The reaction was stirred at 30° C. for 1 h. After finishing the reaction, the solvent was blown away by compressed air. The crude peptide product was precipitated by ethyl ether. The residue was dissolved in 10 mL 20% ACN/H₂O and subjected to preparative HPLC purification (20-60%, over 35 min). Lyophilization afforded 3d as a white powder (5.9 mg, 68%).

### Daptomycin-SCF₃ (3e)

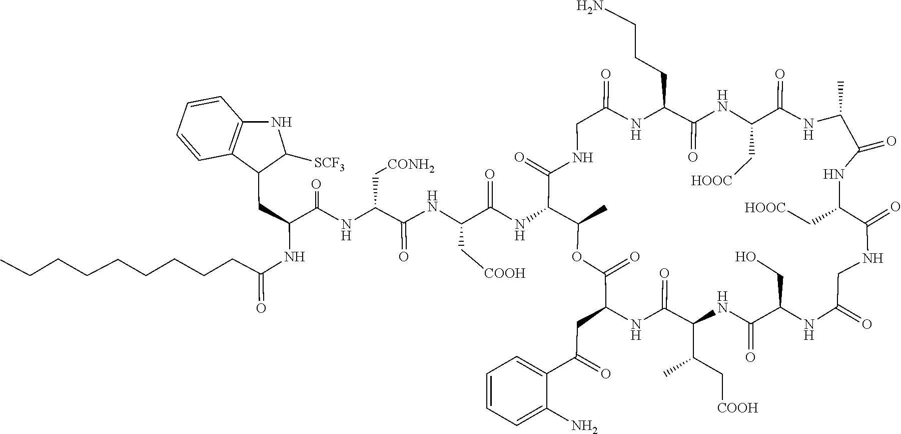

UV (190-400 nm) and MS (300-2000 m/z) traces from UPLC-MS analysis of 3e (gradient 5-95% CH₃CN/H₂O containing 0.1% TFA over 5 min at a flow rate of 0.4 mL/min) are shown in FIG. 8. ESI-MS calcd. for C₇₃H₁₀₀F₃N₁₇O₂₆S: [M+H]⁺ m/z=1721.7, found 1721.0; [M+2H]²⁺ m/z=861.3, found 860.9; [M+3H]³⁺ m/z=580.3, found 580.4.

### Daptomycin-SCF₂H (3f)

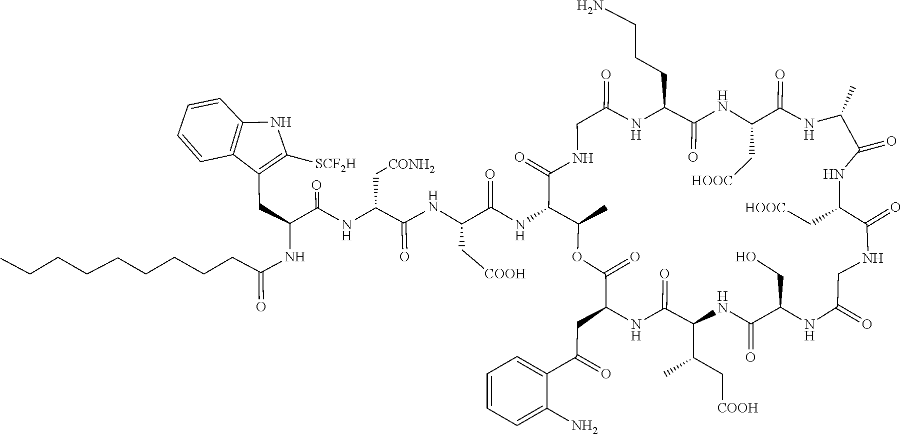

UV (190-400 nm) and MS (250-3000 m/z) traces from UPLC-MS analysis of 3f (gradient 5-95% CH₃CN/H₂O containing 0.1% TFA over 5 min at a flow rate of 0.4 mL/min) are shown in FIG. 9. ESI-MS calcd. for C₇₃H₁₀₁F₂N₁₇O₂₆S: [M+2H]²⁺ m/z=852.3, found 852.2.

### Daptomycin-SMe (3g)

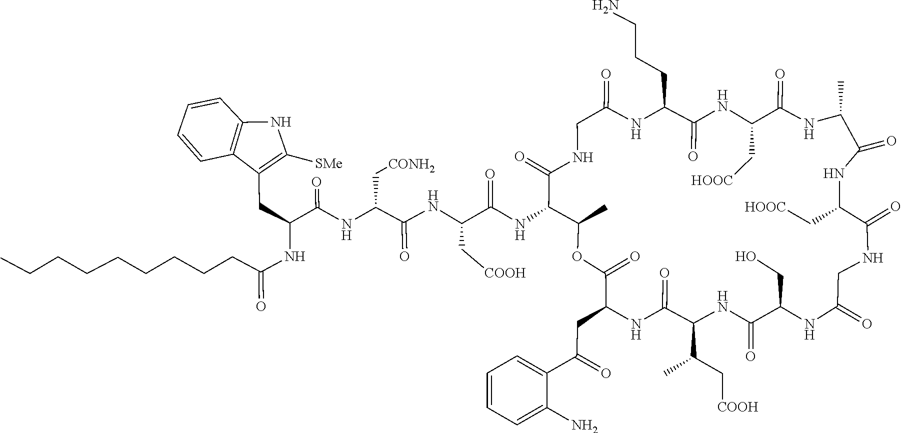 [SMILES: CCCCCCCCCC(=O)N[C@@H](Cc1c(SC)[nH]c2ccccc12)C(=O)N[C@H](CC(N)=O)C(=O)N[C@@H](CC(=O)O)C(=O)CN[C@@H]1C(=O)NCC(=O)N[C@@H](CCCN)C(=O)N[C@@H](CC(=O)O)C(=O)N[C@H](C)C(=O)N[C@@H](CC(=O)O)C(=O)NCC(=O)N[C@H](CO)C(=O)N[C@@H]([C@@H](C)CC(=O)O)C(=O)N[C@@H](CC(=O)c2ccccc2N)C(=O)O[C@@H]1C]

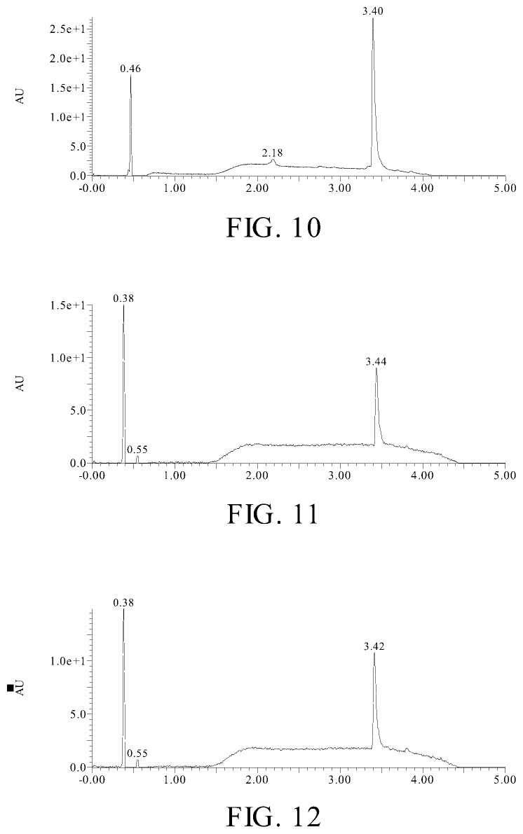

UV (190-400 nm) and MS (250-3000 m/z) traces from UPLC-MS analysis of 3g (gradient 5-95% CH₃CN/H₂O containing 0.1% TFA over 5 min at a flow rate of 0.4 mL/min) are shown in FIG. 10. ESI-MS calcd. for C₇₃H₁₀₃N₁₇O₂₆S: [M+2H]²⁺ m/z=834.3, found 834.3.

### Semaglutide-SCF₃ (3k)

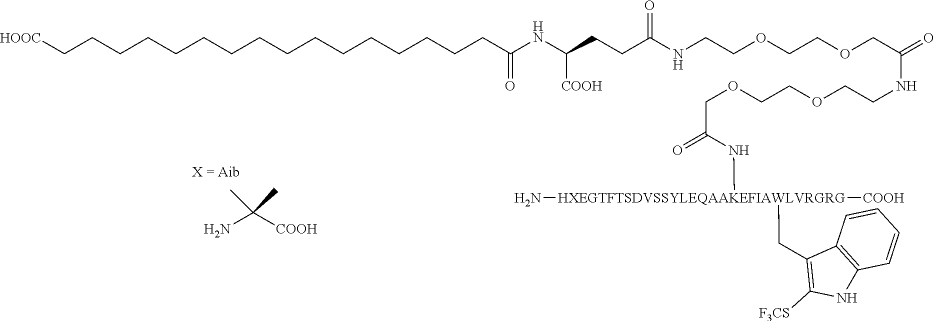

UV (190-400 nm) and MS (250-3000 m/z) traces from UPLC-MS analysis of 3k (gradient 5-95% CH₃CN/H₂O containing 0.1% TFA over 5 min at a flow rate of 0.4 mL/min) are shown in FIG. 11. ESI-MS calcd. for C₁₈₈H₂₉₀F₃N₄₅O₅₉S: [M+2H]²⁺ m/z=2107.8, found 2108.1; [M+3H]3+ m/z=1405.5, found 1405.7; [M+4H]⁴⁺ m/z=1054.4, found 1054.8.

### Semaglutide-SCF₂H (31)

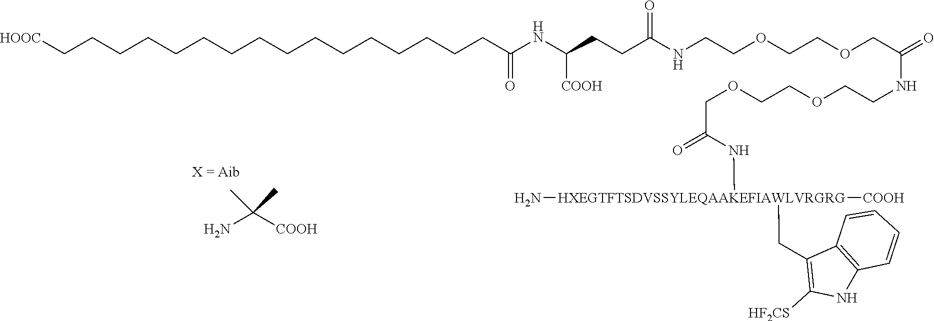

UV (190-400 nm) and MS (250-3000 m/z) traces from UPLC-MS analysis of 31 (gradient 5-95% CH₃CN/H₂O containing 0.1% TFA over 5 min at a flow rate of 0.4 mL/min) are shown in FIG. 12. ESI-MS calcd. for C₁₈₈H₂₉₁F₂N₄₅O₅₉S: [M+2H]²⁺ m/z=2098.8, found 2098.6; [M+3H]³⁺ m/z=1399.5, found 1399.7; [M+4H]⁴ m/z=1049.9, found 1050.1.

### Semaglutide-SMe (3m)

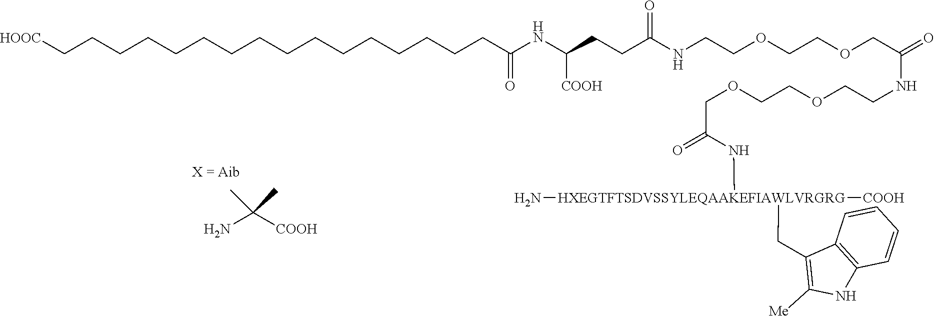

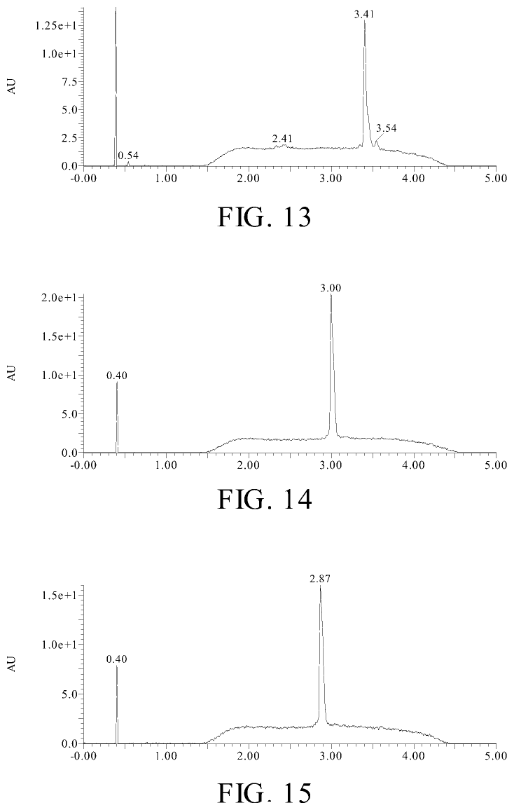

UV (190-400 nm) and MS (250-3000 m/z) traces from UPLC-MS analysis of 3m (gradient 5-95% CH₃CN/H₂O containing 0.1% TFA over 5 min at a flow rate of 0.4 mL/min) are shown in FIG. 13. ESI-MS calcd. for C₁₈₈H₂₉₃N₄₅O₅₉S: [M+2H]²⁺ m/z=2080.8, found 2081.6; [M+3H]³⁺ m/z=1387.5, found 1387.7; [M+4H]⁴⁺ m/z=1040.9, found 1040.8; [M+5H]5+ m/z=832.9, found 832.6; [M+6H]6+ m/z=694.2, found 694.4.

### Triptorelin-SCF₃ (3t)

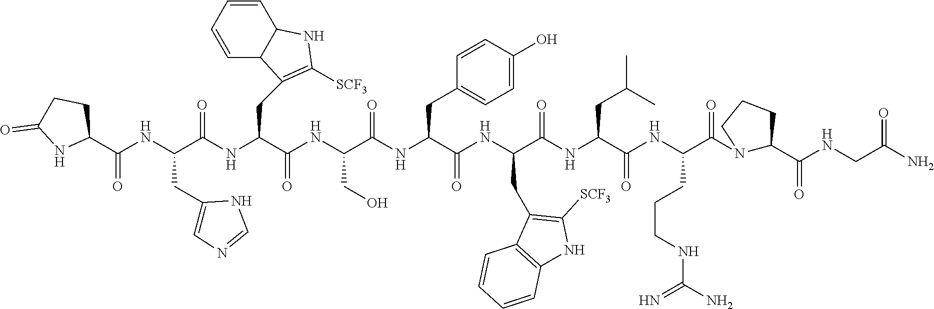

UV (190-400 nm) and MS (250-3000 m/z) traces from UPLC-MS analysis of 3t (gradient 5-95% CH₃CN/H₂O containing 0.1% TFA over 5 min at a flow rate of 0.4 mL/min) are shown in FIG. 14. ESI-MS calcd. for C₆₆H₈OF₆N₁₈O₁₃S₂: [M+2H]²⁺ m/z=756.7, found 756.9.

### Triptorelin-SCF₃ (3u)

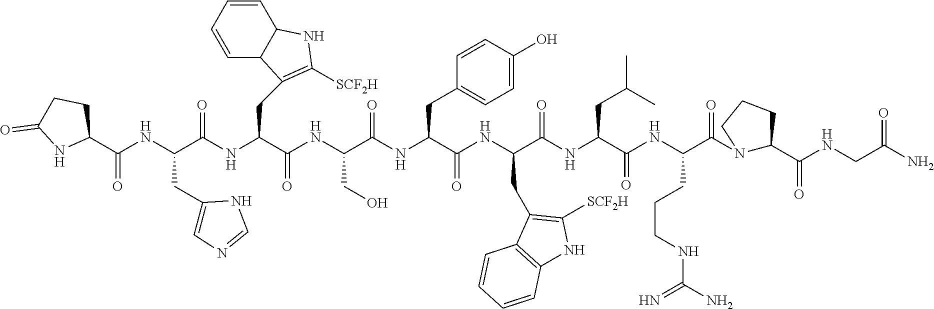

UV (190-400 nm) and MS (250-3000 m/z) traces from UPLC-MS analysis of 3u (gradient 5-95% CH₃CN/H₂O containing 0.1% TFA over 5 min at a flow rate of 0.4 mL/min) are shown in FIG. 15. ESI-MS calcd. for C₆₆H₈₂F₄N₁₈O₁₃S₂: [M+2H]²⁺ m/z=738.8, found 738.9.

1. A method of late-stage modification of a peptide comprising:
(i) maintaining a reaction mixture at a sufficient temperature for a sufficient period of time to form a product,
wherein the reaction mixture comprises the peptide comprising one or more tryptophan residues, a sulfynyl reagent, and a solvent,
and wherein the product comprises a sulfenylated peptide, and the sulfenylation is on at least one of the one or more tryptophan residues of the peptide.

2. The method of claim 1, wherein the peptide is:
(a) linear or cyclic; and/or
(b) a random peptide or a peptide drug.

3. (canceled)

4. The method of claim 1, wherein the peptide comprises natural amino acids.

5. The method of claim 1, wherein the sulfynyl reagent is a thiosulfonate reagent having the structure of: 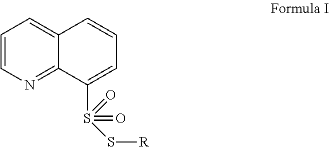
wherein R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, a hydroxyl, an azido, an alkyl, an alkynyl, an alkoxy, a carbonyl (e.g., carboxyl), a nitro, or a cyano.

6. The method of claim 1, wherein the solvent is trifluoroacetic acid, optionally wherein the trifluoroacetic acid acts as catalyst and/or is the only solvent in the reaction mixture.

7. (canceled)

8. The method of claim 1, wherein the reaction mixture is maintained at a temperature ranging from 20° C. to 35° C., such as about 30° C., for a time period up to 1 hour, up to 2 hours, up to 3 hours, ranging from 10 mins to 1 hour, from 20 mins to 2 hours, or from 30 mins to 3 hours.

9. The method of claim 1, wherein the peptide and the sulfynyl reagent have a molar ratio (peptide:sulfynyl reagent) ranging from 0.1 to 1, such as about 0.2.

10. The method of claim 1, wherein the peptide has a structure of: 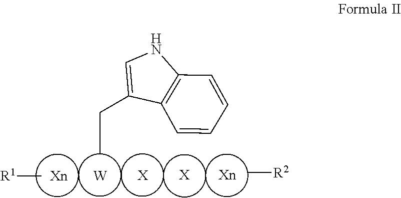
and the sulfenylated peptide has a structure of: 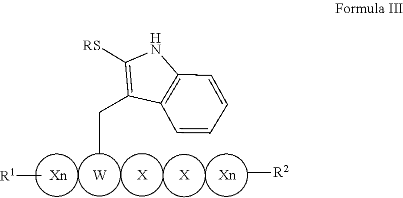
wherein each n is independently 0 or a positive integer (such as an integer from 1 to 50); R₁ is an amino group (such as —NH₂ or NHAc); R₂ is a carbonyl group (such as —COOH) or an amido group (such as —CONH₂); each X is independently a natural amino acid residue (including L- and D-configurations); R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, hydroxyl, an azido, an alkyl, an alkynyl, an alkoxy, a carbonyl (e.g., carboxyl), a nitro, or a cyano; and 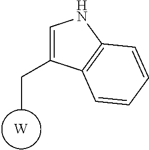
represents a L- or D-tryptophan residue.

11. The method of claim 10, wherein X is not a tryptophan residue.

12. The method of claim 1, wherein the peptide is a peptide drug, and the sulfenylated peptide is the peptide drug comprising one or more sulfenylated tryptophan residues represented by 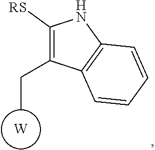
wherein R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, a hydroxyl, an azido, an alkyl, an alkynyl, an alkoxy, a carbonyl (e.g., carboxyl), a nitro, or a cyano,
Optionally wherein the peptide drug is octreotide, lanreotide, setmelanotide, somatostain, semaglutide, leuprolide, histrelin, triptorelin, goserelin, melltin, or daptomycin.

13. (canceled)

14. (canceled)

15. The method of claim 1, wherein the sulfenylated peptide has any one of the following structures:

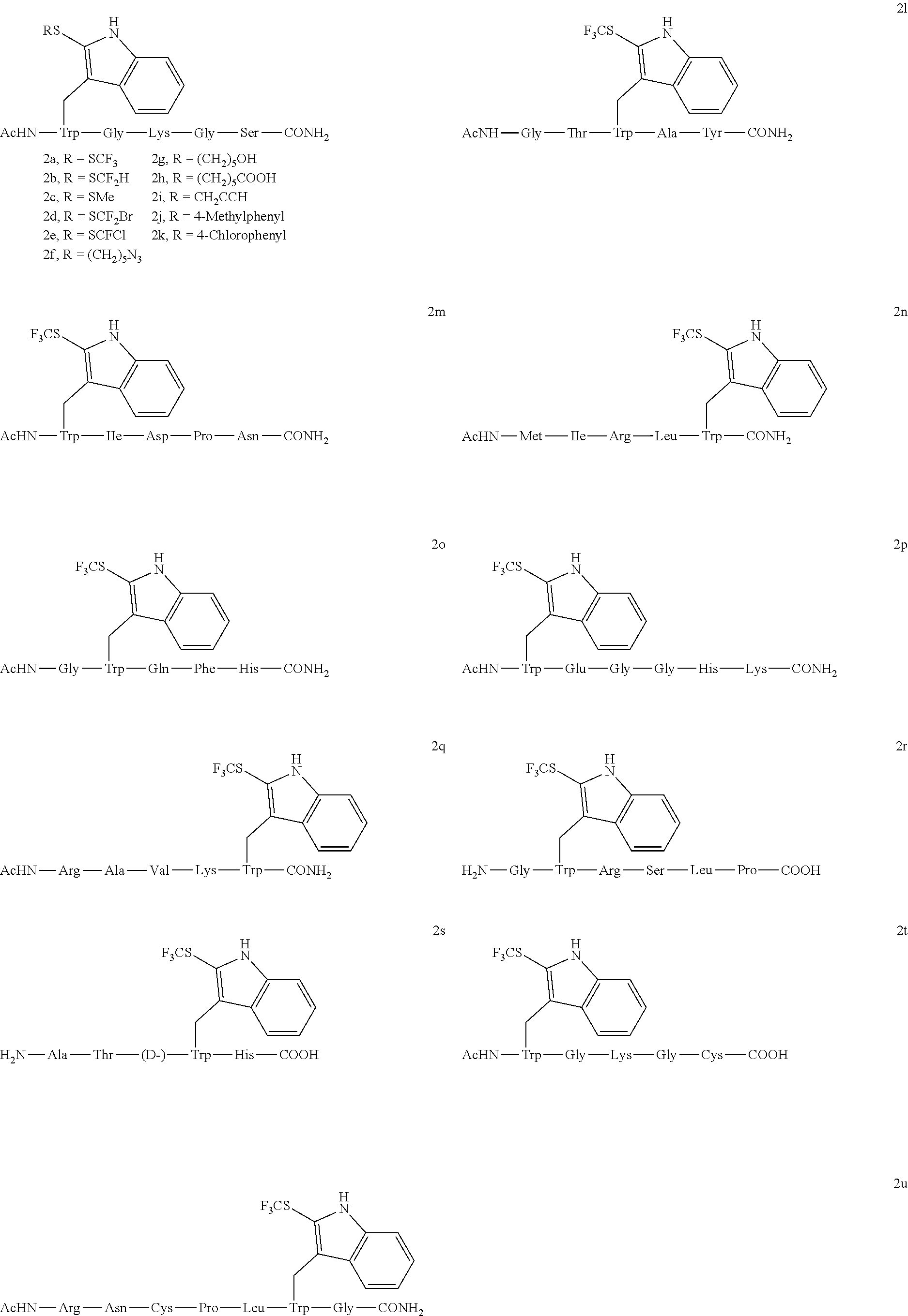

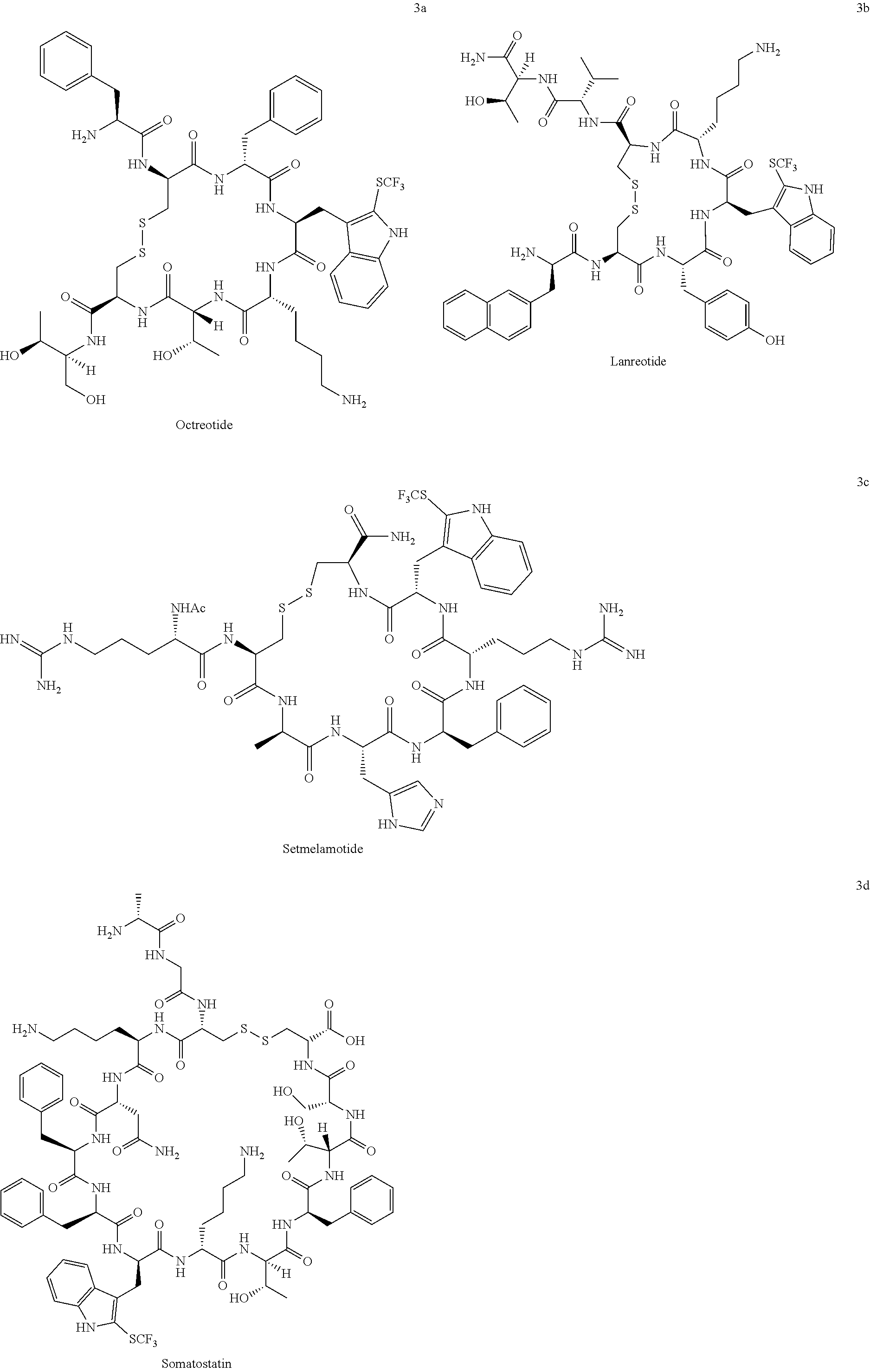

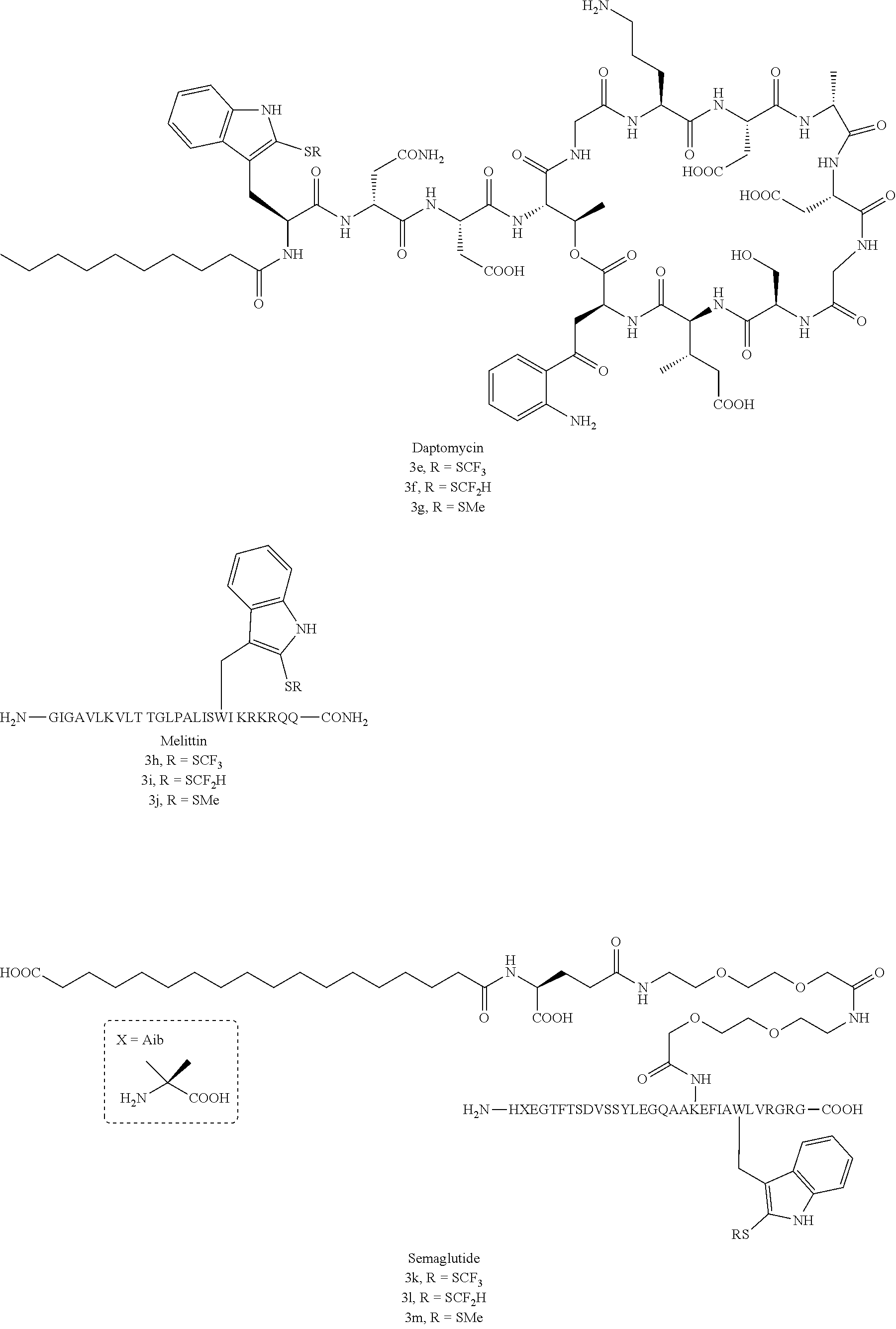

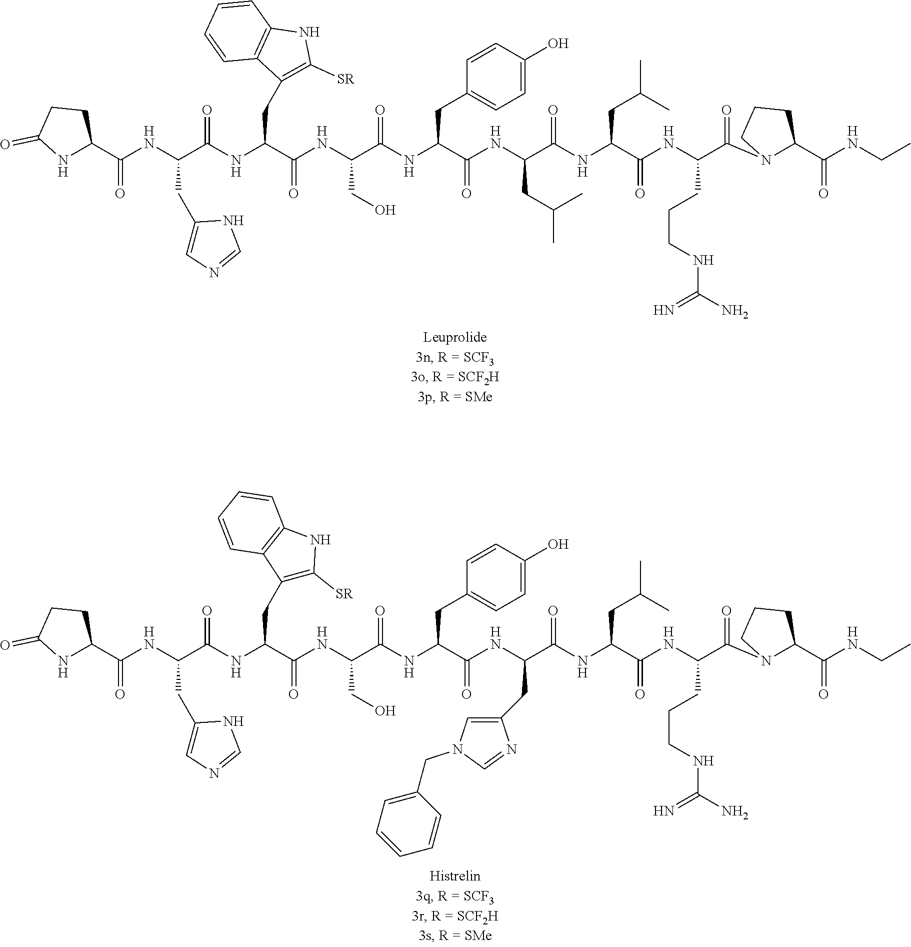

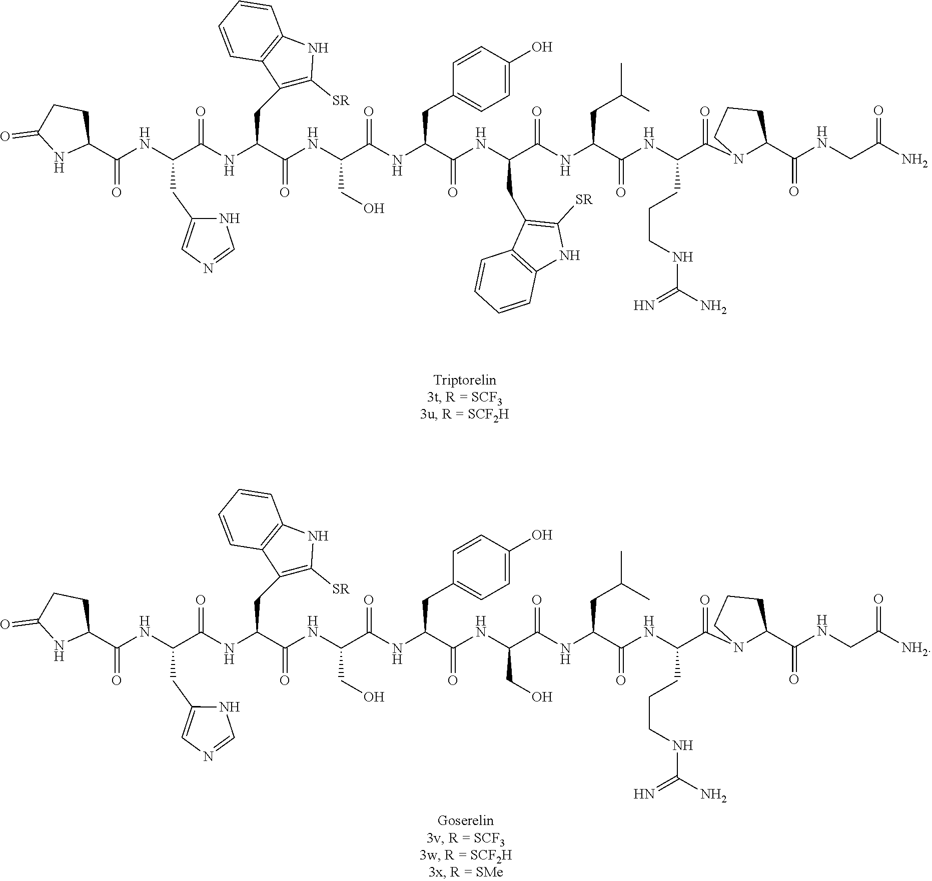

16. The method of claim 1, further comprising stirring the reaction mixture during step (i), drying the product to remove the solvent, purifying the product, and/or lyophilizing the sulfenylated peptide,
optionally wherein the product is purified by precipitating the product using a precipitation solvent (such as ethyl ether) to obtain a precipitate, dissolving the precipitate in a separation solvent (such as a mixture of ACN and water) to obtain a solution, and subjecting the solution to high-performance liquid chromatograph to separate the sulfenylated peptide from impurities.

17. (canceled)

18. The method of claim 1, wherein the sulfenylated peptide has a yield of at least 50%, at least 55%, at least 60%, at least 70%, or at least 80%.

19. A sulfenylated peptide comprising one or more sulfenylated tryptophan residues represented by 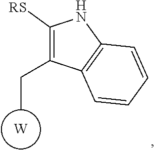
wherein R is a haloalkyl, a substituted or unsubstituted alkyl, a substituted or unsubstituted alkenyl, a substituted or unsubstituted alkynyl, a substituted or unsubstituted aryl, a substituted or unsubstituted polyaryl, a substituted or unsubstituted heteroaryl, a substituted or unsubstituted heteropolyaryl, a substituted or unsubstituted heterocyclic, and wherein the substituents, when present, is a halogen, hydroxyl, an azido, an alkyl, an alkynyl, an alkoxy, a carbonyl (e.g., carboxyl), a nitro, or a cyano.

20. The sulfenylated peptide of claim 19, wherein the peptide is:
(a) a linear or cyclic peptide; and/or
(b) a random peptide or a peptide drug.

21. (canceled)

22. The sulfenylated peptide of claim 19, having a structure of: 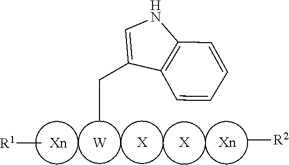
wherein each n is independently 0 or a positive integer (such as an integer from 1 to 50); R₁ is an amino group (such as —NH₂ or NHAc); R₂ is a carbonyl group (such as —COOH) or an amido group (such as —CONH₂); each X is independently a natural amino acid residue (including L- and D-configurations); and 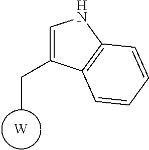
represents a L- or D-tryptophan residue.

23. The sulfenylated peptide of claim 22, wherein X is not a tryptophan residue.

24. The sulfenylated peptide of claim 19, wherein the peptide is a peptide drug, optionally wherein the peptide drug is octreotide, lanreotide, setmelanotide, somatostain, semaglutide, leuprolide, histrelin, triptorelin, goserelin, melltin, or daptomycin.

25. The sulfenylated peptide of claim 19, wherein R is —CF₃, —CF₂H, —CF₂Br, —CHFCl, —CF₂-alkyl, —CHF-alkyl, a substituted or unsubstituted alkyl (e.g., a substituted or unsubstituted C1-C6 alkyl, a substituted or unsubstituted C1-C5 alkyl, a substituted or unsubstituted C1-C4 alkyl, or a substituted or unsubstituted C1-C3 alkyl), a substituted or unsubstituted alkynyl (e.g., —CH₂CCH), or a substituted or unsubstituted aryl (e.g., a substituted or unsubstituted phenyl), and wherein the substituent(s), when present, is a halogen, a hydroxyl, a carboxyl, an azido, an alkyl (e.g., an unsubstituted C1-C6 alkyl, for example, methyl, ethyl, propyl, or butyl), an alkyne, or an alkoxy.

26. The sulfenylated peptide of claim 19, having any one of the following structures: 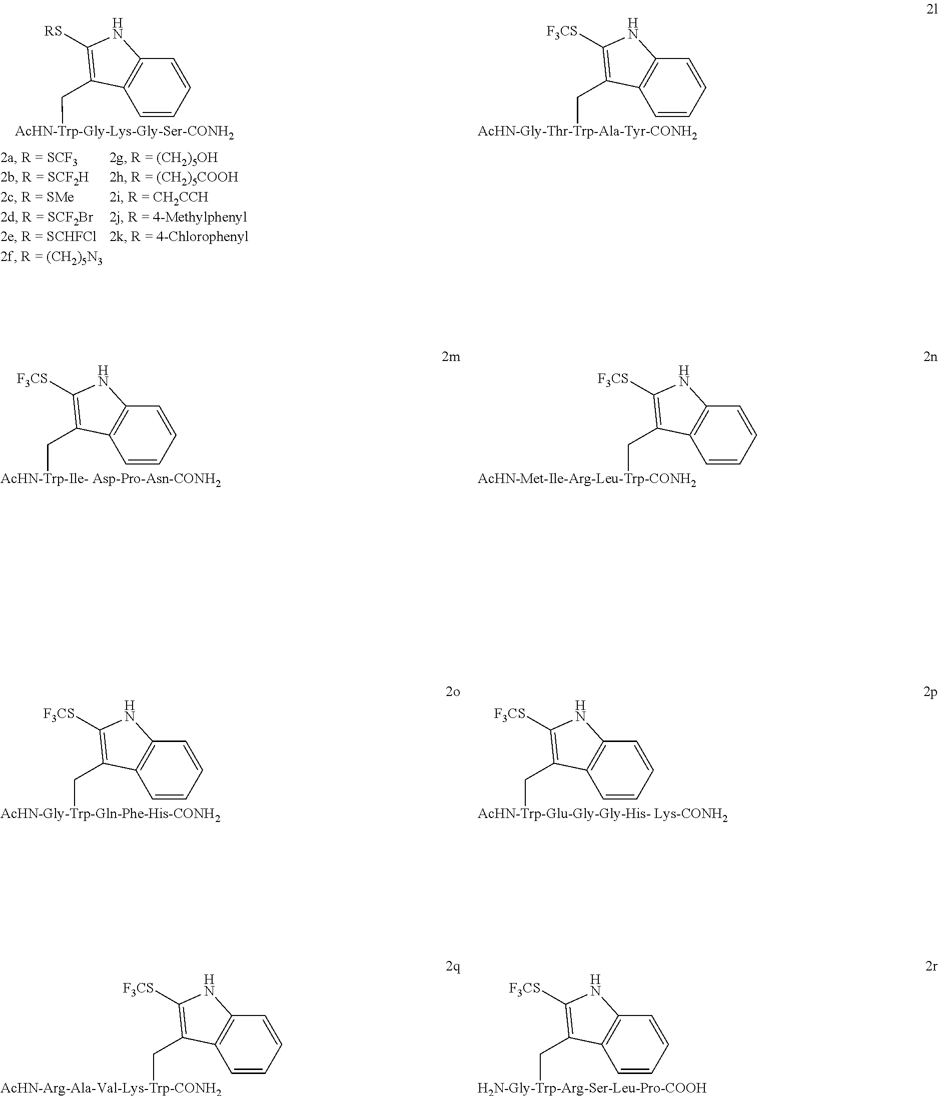 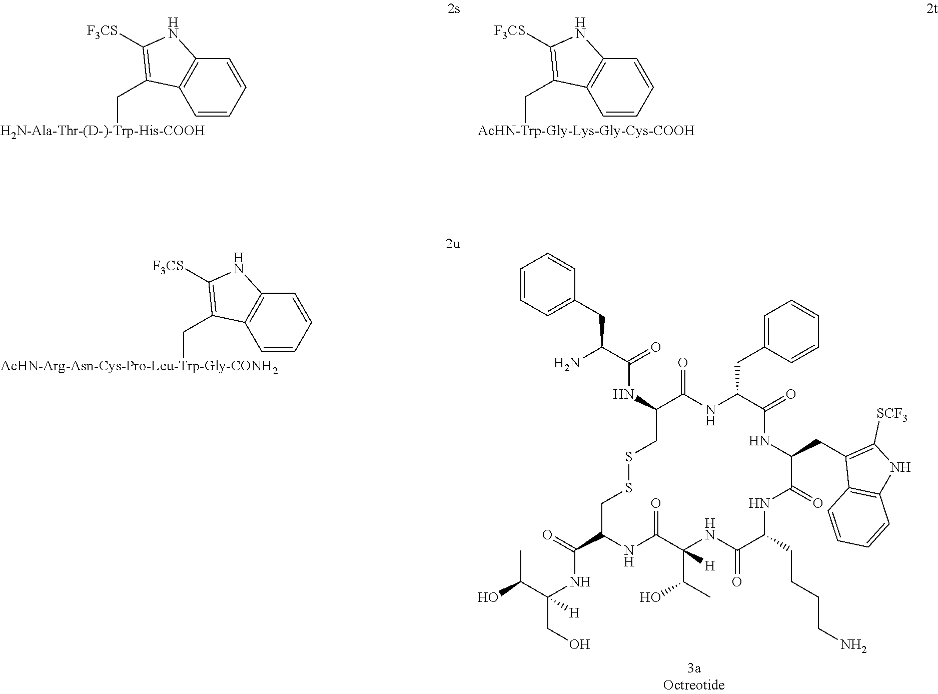 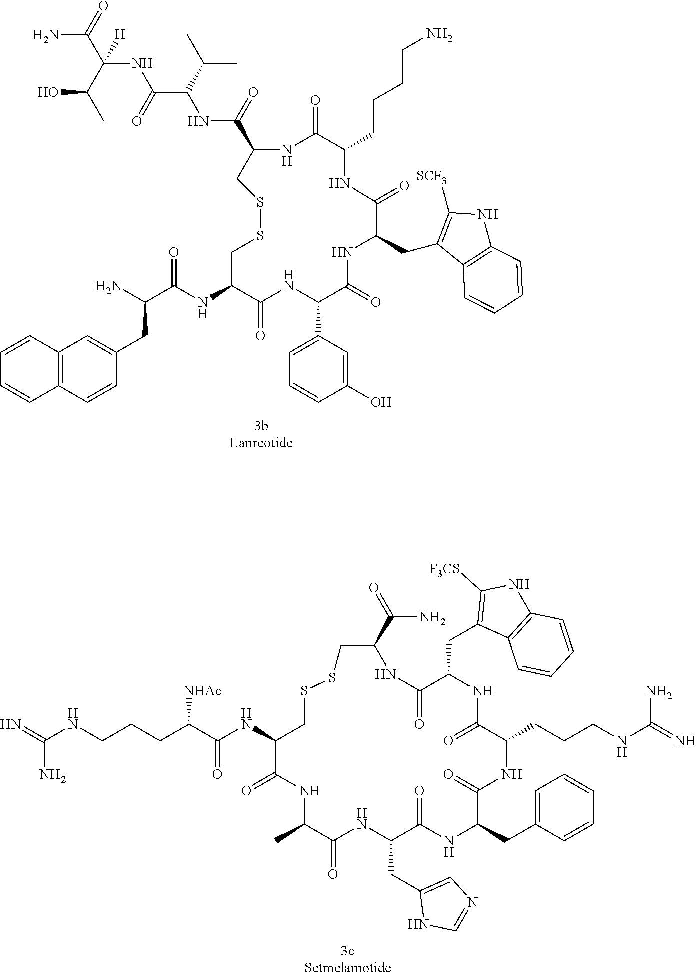 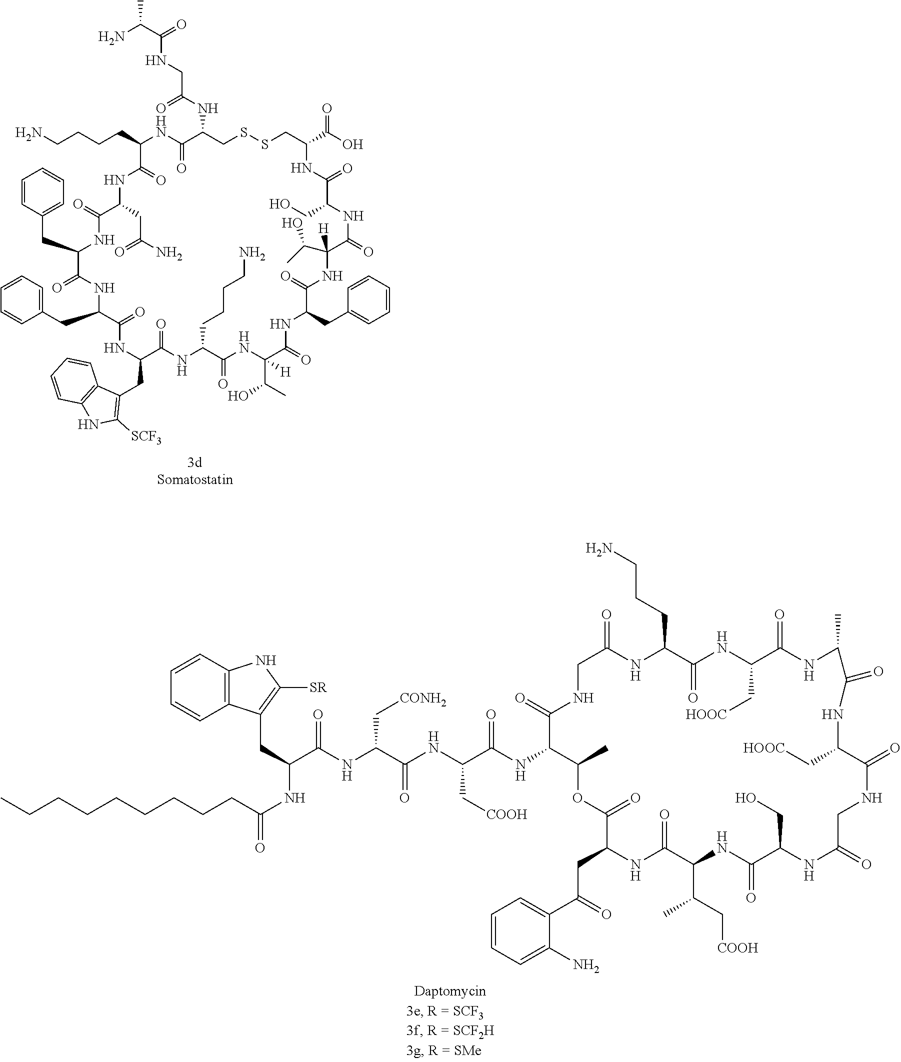 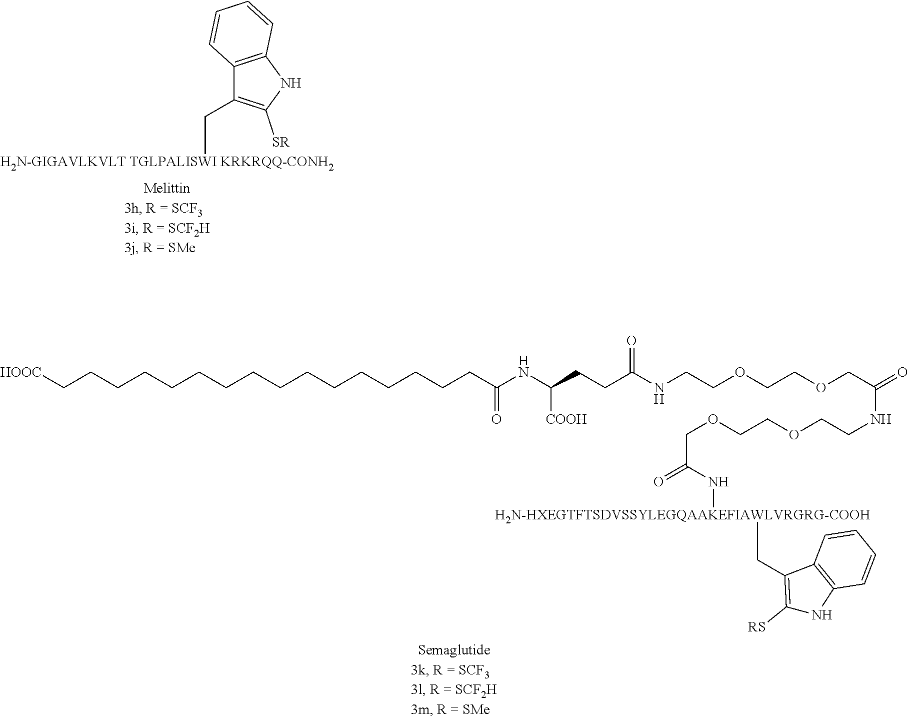 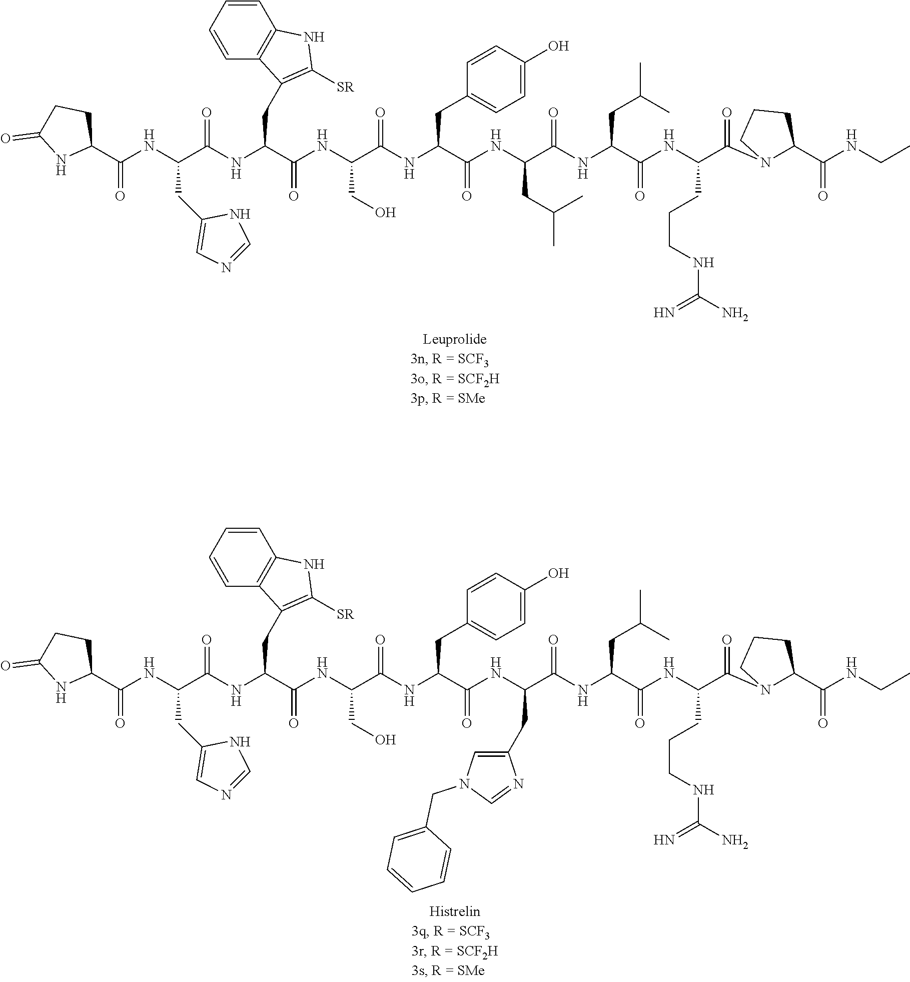 [chemical structure]
